(19) United States
(12) Patent Application Publication
(10) Pub. No.: US 2022/0415434 A1
(43) Pub. Date: Dec. 29, 2022

(54) METHODS FOR CANCER CELL STRATIFICATION

(71) Applicants: GARVAN INSTITUTE OF MEDICAL RESEARCH, Darlinghurst (AU); THE UNIVERSITY OF NORTH CAROLINA AT CHAPEL HILL, Chapel Hill, NC (US)

(72) Inventors: Charles Perou, Chapel Hill, NC (US); Daniel Lee Roden, Darlinghurst, NSW (AU); Sunny Ziyang Wu, Darlinghurst, NSW (AU); Aatish Thennaven, Chapel Hill, NC (US); Alex Swarbrick, Darlinghurst, NSW (AU); Ghamdan Abdulqawi Al-Eryani, Darlinghurst (AU)

(21) Appl. No.: 17/849,476

(22) Filed: Jun. 24, 2022

(30) Foreign Application Priority Data
Jun. 25, 2021 (AU) 2021901929

(51) Int. Cl. G16B 20/00; C12Q 1/6886; G16B 40/20

(52) U.S. Cl. CPC G16B 20/00; C12Q 1/6886; G16B 40/20; C12Q 2600/158; C12Q 2600/156; C12Q 1/6869

(57) ABSTRACT

The present invention relates to methods for the classification and stratification of cells within tumours. In one aspect, the invention provides methods for classifying cancer cells into intrinsic cancer subtypes, as well as for diagnosing, prognosing and evaluating a response to therapy for patients afflicted with cancer.

### CROSS-REFERNCE TO RELATED APPLICATIIONS

This application claims the benefit of and priority from Australian Provisional Application No. 2021901929, filed Jun. 25, 2021, the contents and disclosures of which are incorporated herein by reference in their entirety.

### TECHNICAL FIELD

The present invention relates to methods for the classification and stratification of cells within tumours. In one aspect, the invention provides methods for classifying cancer cells into intrinsic cancer subtypes, as well as for diagnosing, prognosing and evaluating a response to therapy for patients afflicted with cancer.

This invention was made with government support under Grant Numbers CA058223 and CA148761 awarded by the National Institutes of Health and The Breast Cancer Research Foundation. The government has certain rights in the invention.

### BACKGROUND ART

Cancer largely results from various molecular aberrations comprising somatic mutational events such as single nucleotide mutations, copy number changes and DNA methylations. In addition, cancer is viewed as a wildly heterogeneous disease, consisting of different subtypes with diverse molecular progression of oncogenesis and therapeutic responses. Many organ-specific cancers have established definitions of molecular subtypes on the basis of genomic, transcriptomic, and epigenomic characterizations, indicating diverse molecular oncogenic processes and clinical outcomes.

One such example is breast cancer (BrCa), which is stratified based on the expression of the estrogen receptor (ER), progesterone receptor (PR) and overexpression of HER2 or amplification of the HER2 gene ERBB2. This results in three broad clinical subtypes of BrCa: Luminal (ER+, PR+/−), HER2+ (HER2+, ER+/−, PR+/−) and triple negative (TNBC; ER−, PR−, HER2−) that correlate with prognosis and define treatment strategies. Luminal cancers have an inherently less aggressive natural history than the Her2+ and TNBC subsets and are typically treated with systemic endocrine therapy targeting the Estrogen Receptor+/−cytotoxic chemotherapy. Her2+ cancers are treated with small molecule and antibody-based systemic drugs targeting the Her2 receptor plus cytotoxic chemotherapy. TNBC are typically only eligible for systemic cytotoxic chemotherapy and thus have the poorest outcomes of the 3 subtypes. BrCa are also stratified based on bulk transcriptomic profiling using the ‘PAM50’ gene signature into five ‘intrinsic’ molecular subtypes: luminal-like (LumA and LumB), HER2-enriched (HER2E), basal-like (BLBC) and normal-like. There is ˜70-80% concordance between molecular subtypes and clinical subtypes. For instance, the HER2E subtype is composed of clinically HER2+ and HER2− BrCa, as well as those that are ER+ and ER−3. PAM50 has provided important insights into prognosis and treatment, however this method is based on the analysis of whole cancer tissue samples and does not take into account inherent heterogeneity within cancer cells. Moreover, genes analysed for the PAM50 test are generally very poorly detected when utilising a scRNA-Seq data approach.

Thus, more detailed methods of analysis of various cancers that can accurately characterise a cancer subtype are required. The identification of tumour heterogeneity is essential to the design of effective stratified treatments and for the identification of treatments that can be extended to particular tumour cell types.

In view of the above-described limitations, there is a need for improved methods for cancer stratification that overcome one or more of the above described limitations.

It will be clearly understood that, if a prior art publication is referred to herein, this reference does not constitute an admission that the publication forms part of the common general knowledge in the art in Australia or in any other country.

### SUMMARY OF INVENTION

In an aspect of the invention, the invention provides a method for classifying cancer cells from a test sample into one or more breast cancer intrinsic subtypes, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal);
b) generating, from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3; and
d) generating gene expression signature scores for the test gene expression profile, each gene expression signature score being a comparison between the test gene expression profile and the gene expression signature of a respective breast cancer intrinsic subtype;
   wherein the cancer cells from the test sample are classified into one or more breast cancer intrinsic subtypes based on the highest gene expression signature score, thereby classifying cancer cells from a test sample into one or more breast cancer intrinsic subtypes.

In another aspect of the invention, there is provided a method of generating gene expression signatures for classifying cancer cells into one or more breast cancer intrinsic subtypes, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal); and
b) generating, from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
   wherein:
   a test gene expression profile can be generated from cancer cells isolated from the test sample, wherein the test gene expression profile is based on expression of one or more of the genes listed in Table 3;
   gene expression signature scores can be generated for the test gene expression profile, each gene expression signature score being a comparison between the test gene expression profile and the gene expression signature of a respective breast cancer intrinsic subtype; and
   the cancer cells from the test sample can be classified into one or more breast cancer intrinsic subtypes based on the highest gene expression signature score.

In another aspect of the invention, there is provided a method for classifying cancer cells from a test sample into one or more breast cancer intrinsic subtypes, the method comprising:
a) generating a test gene expression profile from cancer cells isolated from the test sample, wherein the gene expression profile is based on expression of one or more of the genes listed in Table 3; and
b) generating gene expression signature scores for the test gene expression profile, each gene expression signature score being a comparison between the test gene expression profile and a gene expression signature of a respective breast cancer intrinsic subtype,
   wherein:
   a training gene expression profile can be generated from cancer cells that have been isolated from samples classified according to breast cancer intrinsic subtype Luminal A (LumA), Luminal B (LumB), Basal-like (Basal), HER2-enriched (HER2E), or Normal-like (Normal), and
   the gene expression signatures can be generated from the training gene expression profile, the gene expression signatures defining breast cancer intrinsic subtypes Basal Single Cell (Basal SC), HER2-enriched Single Cell (HER2E SC), Luminal A Single Cell (LumA SC) and Luminal B Single Cell (LumB SC), each gene expression signature being based on expression of one or more of the genes listed in Table 3;
   wherein the cancer cells from the test sample are classified into one or more breast cancer intrinsic subtypes based on the highest gene expression signature score.

In an embodiment of the invention, the generation of gene expression signatures from the training gene expression profile comprises using a machine learning algorithm, preferably a supervised algorithm.

In an embodiment, the generation of a gene expression score comprises calculating the average (mean) read counts for each breast cancer intrinsic subtype Basal SC, HER2E SC, LumA SC and LumB SC. In a further embodiment, cells are assigned to the breast cancer intrinsic subtype with the highest signature score.

In an embodiment, the method further comprises identifying a suitable treatment for the subject based on the classification of the cells in the test sample to the cancer intrinsic subtype. In this embodiment, the treatment may comprise chemotherapy, hormonal therapy, radiation therapy, biological therapy such as immunotherapy, small molecule therapy or antibody therapy, or a combination thereof.

In an embodiment of the invention, the cells that make up the major proportion of the cancer intrinsic subtype will determine the type of treatment provided to a subject. In another embodiment, the cells that make up the minor proportion of the cancer intrinsic subtype will determine the type of treatment provided to a subject.

In another aspect, the invention provides a method for diagnosing a breast cancer in a test sample from a subject, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3; and
d) generating gene expression signature scores for the test gene expression profile, each gene expression signature score being a comparison between the test gene expression profile and the gene expression signature of a respective breast cancer intrinsic subtype;
   wherein the cancer cells from the test sample are classified into one or more breast cancer intrinsic subtypes based on the highest gene expression signature score, and
   wherein the proportions of cells isolated from the test sample and classified into to the breast cancer intrinsic subtypes is determinative of the diagnosis of breast cancer in the subject,
   thereby diagnosing a breast cancer in the subject.

In an embodiment of this aspect, the breast cancer clinical subtype is diagnosed as substantially HR+/HER2− (“Luminal A”); HR−/HER2− (“Triple Negative”); HR+/HER2+ (“Luminal B”) or HR−/HER2+ (“HER2-enriched”). In another embodiment, the subject has been diagnosed previously with a non-invasive or invasive carcinoma including ductal, lobular colloid (mucinous), medullary, micropapillary, papillary, and tubular invasive carcinoma.

In another embodiment, the subject from which the sample was obtained may exhibit one or more of the following symptoms:
presence of a lump in the breast or underarm;
thickening or swelling of part of the breast;
irritation or dimpling of breast skin;
redness or flaky skin in the nipple area or the breast;
pulling in of the nipple or pain in the nipple area;
nipple discharge including blood;
any change in the size or the shape of the breast; and
pain in an area of the breast.

In another embodiment, the method further comprises identifying a suitable treatment for the subject based on the diagnosis of the cancer. In an embodiment, the treatment may comprise one or more of:
surgery;
chemotherapy;
hormonal therapy;
biological therapy such as immunotherapy, small molecule therapy or antibody therapy; and
radiation therapy.

In another embodiment, the method comprises one or more of the following additional diagnostic tests:
breast ultrasound;
diagnostic mammogram;
magnetic resonance imaging (MRI); and
biopsy.

In another aspect, the invention provides a method for prognosing breast cancer in a test sample from a subject, the method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) calculating a risk score for the cells of each of the samples and stratifying the risk scores into higher and lower risk groups;
d) generating a test gene expression profile from cancer cells isolated from the test sample, wherein the test gene expression profile is based on expression of one or more of the genes listed in Table 3;
e) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score;
f) generating a risk score for the cells isolated from the test sample based on the gene expression signature scores; and
g) determining whether the test sample falls within a higher or a lower risk group by comparing the risk score assigned in step (f) to the risk score assigned in (c), wherein assignment to a lower risk group indicates a more favourable outcome, and assignment to a higher risk group indicate a less favourable outcome, thereby prognosing breast cancer in a test sample from a subject.

In an embodiment, the prognosis is selected from the group comprising or consisting of breast cancer specific survival, event-free survival, or response to therapy.

In another aspect, the invention provides a method for treating a breast cancer in a subject, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3;
d) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score; and
e) administering a therapeutically effective amount of a treatment to the subject based on the breast cancer intrinsic subtype classification, thereby treating a breast cancer in the subject.

In another aspect, the invention provides a method for treating a breast cancer in a subject, the method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) calculating a risk score for the cells of each of the samples and stratifying the risk scores into higher and lower risk groups;
d) generating a test gene expression profile from cancer cells isolated from the test sample, wherein the test gene expression profile is based on expression of one or more of the genes listed in Table 3;
e) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score;
f) generating a risk score for the cells isolated from the test sample based on the gene expression signature scores; and
g) determining whether the testing set falls within a higher or a lower risk group by comparing the risk score assigned in step (f) to the risk score assigned in (c), wherein assignment to a lower risk group indicates a more favourable outcome, and assignment to a higher risk group indicate a less favourable outcome; and
g) administering a therapeutically effective amount of a treatment to the subject based on the risk group assignment, thereby for treating a breast cancer in the subject.

In another aspect, the invention provides use of a therapy in the preparation of a medicament for treating a breast cancer in a subject, the treatment comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) calculating a risk score for the cells of each of the samples and stratifying the risk scores into higher and lower risk groups;
d) generating a test gene expression profile from cancer cells isolated from the test sample, wherein the gene expression profile is based on expression of one or more of the genes listed in Table 3;
e) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score
f) generating a risk score for the cells isolated from the test sample based on the gene expression signature scores;
g) determining whether the test sample falls within a higher or a lower risk group by comparing the risk score assigned in step (f) to the risk score assigned in (c), wherein assignment to a lower risk group indicates a more favourable outcome, and assignment to a higher risk group indicate a less favourable outcome; and
h) administering a therapeutically effective amount of a treatment to the subject based on the risk group assignment.

In another aspect, the invention provides use of a therapy in the preparation of a medicament for treating a breast cancer in a subject, the treatment comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the gene expression profile is based on expression of one or more of the genes listed in Table 3;
d) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score; and
e) administering a therapeutically effective amount of a treatment to the subject based on the breast cancer intrinsic subtype assignment.

In an embodiment of any aspect, the risk score is generated by calculating the proportion of basal-like or HER2+ cells in ER+ cancers whereby a higher proportion of these cells is indicative of a poor prognosis.

In another aspect, the invention provides a method of predicting a response to a therapy in a test sample from a subject having breast cancer comprising classifying said subject according to a method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3; and
d) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score,
   wherein the intrinsic tumour subtype is indicative of response to the therapy, thereby predicting a response to a therapy in a subject having breast cancer.

In an embodiment, the therapy comprises an adjuvant or neoadjuvant therapy. In another embodiment, the neoadjuvant or adjuvant therapy comprises or is selected from the group consisting of radiotherapy, chemotherapy, immunotherapy, biological response modifiers or hormone therapy.

In an embodiment, the method further comprises diagnosing the subject with any type of breast cancer defined herein or known in the art. In another embodiment, the method further comprises a step of treating the subject for a period of time sufficient for a therapeutic response prior to obtaining the sample from the subject.

In an embodiment of any aspect of the invention, the method further comprises providing or being provided with a test sample comprising cancer cells.

In an embodiment of any aspect of the invention, the method further comprises enzymatic dissociation of tumours, preferably using a tumour dissociation kit and isolating the cancer cells from non-cancer cells by flow cytometry using fluorescent antibodies against epithelial and non-epithelial markers. In another embodiment, the isolation of cancer cells from non-cancer cells is performed by generating a CNV signal for individual cells using an inferCNV method with a 100 gene sliding window. In a preferred embodiment, the test gene expression profile is generated from a sample comprising at least 200 cancer cells.

In an embodiment the cancer cells comprise neoplastic epithelial cells. In yet another embodiment, the cancer cells are derived from a sample from a subject with a non-invasive or invasive carcinoma including ductal, lobular, lobular colloid (mucinous), medullary, micropapillary, papillary, and tubular invasive carcinomas. In yet another embodiment, the samples are untreated breast cancers.

In an embodiment of any aspect of the invention, one or more clinical variables are also assessed including tumour size, node status, histologic grade, estrogen hormone receptor status, progesterone hormone receptor status, HER-2 levels, and tumour ploidy.

In an embodiment of any aspect of the invention, the gene expression profile is generated using reverse transcription and real-time quantitative polymerase chain reaction (qPCR) with primers specific for each of the genes. In another embodiment, the gene expression profile is generated by microarray analysis with probes specific for each of the genes. In a preferred embodiment, the gene expression profile is generated using single cell RNA sequencing or other methods known in the art.

In an embodiment of any aspect of the invention, the gene expression profile is normalised to a control, preferably one or more housekeeping genes. In this embodiment, the housekeeping genes may be selected from RRN18S, ACTB, GAPDH, PGK1, PPIA, RPL13A, RPLPO, B2M, GUSB, HPRT1, TBP.

In a preferred embodiment of any aspect of the invention, the gene expression profile is based on expression of at least 20, 40, 60, 80, 100, 120, 140, 160, 180, 200, 220, 240, 260, 280, 300 or more of the genes listed in Table 3.

In an embodiment of any aspect of the invention, the generation of the gene expression profile for the training set and testing set comprises determining expression of each of the genes listed in Table 3.

In another aspect, the invention provides a kit for classifying a cancer intrinsic subtype in a test sample, the kit comprising reagents for the detection of one or more of the genes listed in Table 3. In an embodiment, the reagents comprise oligonucleotide primers and/or probes sufficient for the detection and/or quantitation of one or more of the intrinsic genes listed in Table 3.

It will be understood that any of the features described herein can be combined in any combination with any one or more of the other features described herein within the scope of the invention.

### BRIEF DESCRIPTION OF DRAWINGS

This patent application contains at least one drawing executed in color. Copies of this patent application with color drawing(s) will be provided by the Office upon request and payment of the necessary fee.

Various embodiments of the invention will be described with reference to the following drawings according to the following.

FIG. 1. Representative H&E images from all 26 breast tumours analysed by scRNA-Seq in this study. Scale bars represent 400 μm.

FIGS. 2A-2G. Cellular composition of primary breast cancers and the identification of malignant epithelial cells. (FIG. 2A) Integrated dataset overview of 130,246 cells analysed by scRNA-Seq. Clusters are annotated for their cell types as predicted using canonical markers and signature-based annotation using Garnett. (FIG. 2B) Log normalized expression of markers for epithelial cells (EPCAM), proliferating cells (MKI67), T-cells (CD3D), myeloid cells (CD68), B-cells (MS4A1), plasmablasts (JCHAIN), endothelial cells (PECAM1) and mesenchymal cells (fibroblasts/perivascular-like; PDGFRB). (FIG. 2C) Relative proportions of cell types highlighting a strong representation of the major lineages across tumors and clinical subtypes. (FIGS. 2D-2F) UMAP visualization of all epithelial cells, from tumours with at least 200 epithelial cells, colored by tumour (FIG. 2D), clinical subtype (FIG. 2E) and inferCNV classification (FIG. 2F). (FIG. 2G) InferCNV heatmaps of all malignant cells grouped by clinical subtypes. Common subtype-specific CNVs and a chr6 artefact reported previously are marked.

FIGS. 3A-3D. Identifying drivers of neoplastic breast cancer cell heterogeneity. (FIG. 3A) Heatmap showing the average expression (scaled) of all cells assigned to each of the four scSubtypes. The top-5 most highly expressed genes in each subtype are shown, and selected others are highlighted. (FIG. 3B) Percentage of neoplastic cells in each tumour that are classified as each of the scSubtypes. Tumour samples are grouped according to their Allcells-pseudobulk classifications (NL=Normal-like). (FIG. 3C) CK5 and ER immunohistochemistry. Insert 1a/b represent CK5−/ER+ areas; Insert 2a/b represent CK5+/ER− areas. (FIG. 3D) Scatter plot of the proliferation scores and Differentiation Scores (DScores) of each neoplastic cell. Individual cancer cells are colored and grouped based on the scSubtype calls. All pairwise comparisons between cells from each scSubtype were significantly different (Wilcox test p<0.001) for both proliferation and DScores.

FIGS. 4A-4B. Single-cell RNA sequencing metrics and non-integrated data of stromal and immune cells. (FIGS. 4A-4B) UMAP visualization of all 71,220 stromal and immune cells without batch correction and data integration. UMAP dimensional reduction was performed using 100 principal components in the Seurat v3 package. Cells are grouped by tumor (FIG. 4A) and major lineage tiers (FIG. 4B) as identified using the Garnett cell classification method.

FIG. 5. Identification of malignant epithelial cells using inferCNV. InferCNV heatmaps showing all epithelial cells and their associated inferCNV based classification for all tumours. For each cell, the normal cell call, copy number alteration (CNA) values, number of unique molecular identifiers (UMIs) and genes per cell are plotted on the right. Normal cell calls were classified as either Normal (green), Unassigned (grey) or Neoplastic (pink). These classifications are derived from a genomic instability score, which is estimated by the inferred changes at each genomic loci, as determined by inferCNV. High UMI and gene metrics in normal cells importantly show that they are not a product of coverage or low sequencing depth.

FIGS. 6A to 6G. (FIG. 6A) Heirarchical Cluster of Allcells-Pseudobulk (Blue) and Ribozero mRNA-Seq (gold) profiles of the patient samples with TCGA patient mRNA-Seq data. (FIG. 6B) Zoomed in view of the basal cluster showing pairing of Allcells-Pseudobulk and Ribozero mRNA-Seq profiles of 2 representative tumours (dashed red boxes) in the present study. (FIG. 6C) Zoomed in view of the luminal cluster showing pairing of Allcells-Pseudobulk and Ribozero mRNA-Seq profiles of 4 representative tumours (dashed blue boxes) in the present study. (FIG. 6D) Heatmap of scSubtype gene sets across the training and test samples in each individual group. Colored outlined boxes highlighting the top expressed genes per group. (FIG. 6E) Barplot representing proportions of scSubtype calls in individual samples. Test dataset samples are highlighted within the golden colored outline. (FIG. 6F) Scatterplot of individual cancer cells plotted according to the Proliferation score (x-axis) and Differentiation—DScore (y-axis). Individual cells are colored based on the scSubtype calls. (FIG. 6G) Scatterplot of individual TCGA BrCa tumours plotted according to the Proliferation score (x-axis) and Differentiation—DScore (y-axis). Individual patients are colored based on the PAM50 subtype calls.

Preferred features, embodiments and variations of the invention may be discerned from the following Description which provides sufficient information for those skilled in the art to perform the invention. The following Description is not to be regarded as limiting the scope of the preceding Summary of the Invention in any way.

### DETAILED DESCRIPTION

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

In the present specification and claims (if any), the word ‘comprising’ and its derivatives including ‘comprises’ and ‘comprise’ include each of the stated integers but does not exclude the inclusion of one or more further integers.

One skilled in the art will recognize many methods and materials similar or equivalent to those described herein, which could be used in the practice of the present invention. The present invention is in no way limited to the methods and materials described.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Cancer largely results from various molecular aberrations comprising somatic mutational events such as single nucleotide mutations, copy number changes and DNA methylations. In addition, cancer is viewed as a wildly heterogeneous disease, consisting of different subtypes with diverse molecular progression of oncogenesis and therapeutic responses. Many organ-specific cancers have established definitions of molecular subtypes on the basis of genomic, transcriptomic, and epigenomic characterizations, indicating diverse molecular oncogenic processes and clinical outcomes.

The inventors show herein for the first time the development of a single cell method, herein described as the intrinsic subtype classification or “scSubtype” which allows for the identification of tumour subtype heterogeneity. In particular, the methods utilize a supervised algorithm to classify samples according to breast cancer intrinsic subtype. The methods described are based on the gene expression profile of a defined subset of intrinsic genes that has been identified herein as superior for classifying breast cancer intrinsic subtypes, and for predicting risk of relapse and/or response to therapy in a subject diagnosed with breast cancer. The subset of genes suitable for forming the gene expression profile are described herein, for instance in Table 3.

This approach provides advantages over previously described approaches including:
it allows for the dissection of a tumour at a cellular resolution which has been previously unattainable;
it is capable of characterising tumours with low cellularity;
the methods can identify small regions of morphologically malignant cells that express markers that are otherwise different to the markers expressed by the majority of the cells within the tumour; and
analysis of the cancer sample at a cellular resolution provides for an accurate means to predict resistance to particular therapeutics, to predict likely relapse following therapy or to diagnose and/or prognose cancer subtype.

Despite recent advances, the challenge of cancer treatment remains to target specific treatment regimens to distinct tumour types with different pathogenesis, and ultimately personalize tumour treatment in order to maximize outcome. In particular, once a patient is diagnosed with cancer, such as breast cancer, there is a need for methods that allow a practitioner to predict the expected course of disease, including the likelihood of cancer recurrence, long-term survival of the patient and the like, and select the most appropriate treatment options accordingly.

For the purposes of the present invention, “breast cancer” includes, for example, those conditions classified by biopsy or histology as malignant pathology. One of skill in the art will appreciate that breast cancer refers to any malignancy of the breast tissue, including, for example, carcinomas and sarcomas. Particular embodiments of breast cancer include ductal carcinoma in situ (DCIS), lobular carcinoma in situ (LCIS), or mucinous carcinoma. Breast cancer also refers to infiltrating ductal (IDC) or infiltrating lobular carcinoma (ILC). In most embodiments of the invention, the subject of interest is a human patient suspected of or having been diagnosed with breast cancer.

Breast cancer is a heterogeneous disease with respect to molecular alterations and cellular composition. This diversity creates a challenge for researchers trying to develop classifications that are clinically meaningful. Gene expression profiling by microarray has provided insight into the complexity of breast tumours and can be used to provide prognostic information beyond standard pathologic parameters.

Expression profiling of breast cancer identifies biologically and clinically distinct molecular subtypes which may require different treatment approaches. The major intrinsic subtypes of breast cancer referred to as Luminal A, Luminal B, HER2-enriched, Basal-like have distinct clinical features, relapse risk and response to treatment. The “intrinsic” subtypes known as Luminal A (LurnA), Luminal B (LumB), HER2-enriched, Basal-like, and Normal-like were discovered using unsupervised hierarchical clustering of microarray data (Perou et al. (2000) Nature 406:747-752). Intrinsic genes, as described in Perou et al. (2000) Nature 406:747-752, are statistically selected to have low variation in expression between biological sample replicates from the same individual and high variation in expression across samples from different individuals. Thus, intrinsic genes are the classifier genes for breast cancer classification. Although clinical information was not used to derive the breast cancer intrinsic subtypes, this classification has proved to have prognostic significance (Sorlie et al. (2001) PNAS 98(19) 10869-10874).

Breast tumours of the “Luminal” subtype are ER positive and have a similar keratin expression profile as the epithelial cells lining the lumen of the breast ducts (Taylor Papadimitriou et al. (1989) J Cell Sci 94:403-413; Perou et al (2000) New Technologies for Life Sciences: A Trends Guide 67-7 6)). Conversely, ER-negative tumours can be broken into two main subtypes, namely those that overexpress (and are DNA amplified for) HER-2 and GRB7 (HER-2-enriched) and “Basal-like” tumours that have an expression profile similar to basal epithelium and express Keratin 5, 6B, and 17. Both these tumour subtypes are aggressive and typically more deadly than Luminal tumours; however, there are subtypes of Luminal tumours with different outcomes. The Luminal tumours with poor outcomes consistently share the histopathological feature of being higher grade and the molecular feature of highly expressing proliferation genes.

### Clinical Variables

The methods described herein may be further combined with information on clinical variables to aid diagnosis or prognosis, to predict response to treatment or for use in any other method described herein.

As described herein, a number of clinical and prognostic breast cancer factors are known in the art and are used to predict treatment outcome and the likelihood of disease recurrence. Such factors include, for example, lymph node involvement, tumour size, histologic grade, estrogen and progesterone hormone receptor status, HER-2 levels, and tumour ploidy.

In one embodiment, risk of relapse score is provided for a subject diagnosed with or suspected of having breast cancer. This score uses the methods described herein in combination with clinical factors of lymph node status (N) and tumour size (T). Assessment of clinical variables is based on the American Joint Committee on Cancer (AJCC) standardized system for breast cancer staging. In this system, primary tumour size is categorized on a scale of 0-4 (TO: no evidence of primary tumour; T1: ˜2 cm; T2: >2 cm-˜5 cm; T3: >5 cm; T4: tumour of any size with direct spread to chest wall or skin). Lymph node status is classified as N0-N3 (NO: regional lymph nodes are free of metastasis; N1: metastasis to movable, same-side axillary lymphnode(s); N2: metastasis to same-side lymph node(s) fixed to one another or to other structures; N3: metastasis to same-side lymph nodes beneath the breastbone).

Methods of identifying breast cancer patients and staging the disease are well known and may include manual examination, biopsy, review of patient's and/or family history, and imaging techniques, such as mammography, magnetic resonance imaging (MRI), and positron emission tomography (PET). It will be understood that breast cancer stage is usually expressed as a number on a scale of 0 through IV—with stage 0 describing non-invasive cancers that remain within their original location and stage IV describing invasive cancers that have spread outside the breast to other parts of the body.

Stage 0 is used to describe non-invasive breast cancers, such as DCIS (ductal carcinoma in situ). In stage 0, there is no evidence of cancer cells or non-cancerous abnormal cells breaking out of the part of the breast in which they started, or getting through to or invading neighbouring normal tissue. Stage I describes invasive breast cancer (cancer cells are breaking through to or invading normal surrounding breast tissue). Stage IA describes invasive breast cancer in which the tumour measures up to 2 centimeters (cm) and the cancer has not spread outside the breast; no lymph nodes are involved. Stage IB describes invasive breast cancer in which there is no tumour in the breast; instead, small groups of cancer cells—larger than 0.2 millimeter (mm) but not larger than 2 mm—are found in the lymph nodes or there is a tumour in the breast that is no larger than 2 cm, and there are small groups of cancer cells—larger than 0.2 mm but not larger than 2 mm—in the lymph nodes.

Stage II is divided into subcategories known as IIA and IIB. Stage IIA describes invasive breast cancer in which no tumour can be found in the breast, but cancer (larger than 2 millimeters [mm]) is found in 1 to 3 axillary lymph nodes (the lymph nodes under the arm) or in the lymph nodes near the breast bone (found during a sentinel node biopsy) or the tumour measures 2 centimeters (cm) or smaller and has spread to the axillary lymph nodes or the tumour is larger than 2 cm but not larger than 5 cm and has not spread to the axillary lymph nodes. Stage IIB describes invasive breast cancer in which the tumour is larger than 2 cm but no larger than 5 centimeters; small groups of breast cancer cells—larger than 0.2 mm but not larger than 2 mm—are found in the lymph nodes or the tumour is larger than 2 cm but no larger than 5 cm; cancer has spread to 1 to 3 axillary lymph nodes or to lymph nodes near the breastbone (found during a sentinel node biopsy) or the tumour is larger than 5 cm but has not spread to the axillary lymph nodes.

Stage III is divided into subcategories known as IIIA, IIIB, and IIIC. In general, stage IIIA describes invasive breast cancer in which either no tumour is found in the breast or the tumour may be any size; cancer is found in 4 to 9 axillary lymph nodes or in the lymph nodes near the breastbone (found during imaging tests or a physical exam) or the tumour is larger than 5 centimeters (cm); small groups of breast cancer cells (larger than 0.2 millimeter [mm] but not larger than 2 mm) are found in the lymph nodes or the tumour is larger than 5 cm; cancer has spread to 1 to 3 axillary lymph nodes or to the lymph nodes near the breastbone (found during a sentinel lymph node biopsy). Stage IIIB describes invasive breast cancer in which the tumour may be any size and has spread to the chest wall and/or skin of the breast and caused swelling or an ulcer and may have spread to up to 9 axillary lymph nodes or may have spread to lymph nodes near the breastbone. Stage IIIC describes invasive breast cancer in which there may be no sign of cancer in the breast or, if there is a tumour, it may be any size and may have spread to the chest wall and/or the skin of the breast and the cancer has spread to 10 or more axillary lymph nodes or the cancer has spread to lymph nodes above or below the collarbone or the cancer has spread to axillary lymph nodes or to lymph nodes near the breastbone.

Stage IV describes invasive breast cancer that has spread beyond the breast and nearby lymph nodes to other organs of the body, such as the lungs, distant lymph nodes, skin, bones, liver, or brain.

Using the methods of the present invention, the diagnosis and/or prognosis of a breast cancer patient can be determined independent of, or in combination with assessment of these clinical factors. In some embodiments, combining the breast cancer intrinsic subtype classification methods disclosed herein with evaluation of these clinical factors may permit a more accurate risk assessment.

The methods of the invention may be further coupled with analysis of, for example, estrogen receptor (ER) and progesterone receptor (PgR) status, and/or HER-2 expression levels. Other factors, such as patient clinical history, family history and menopausal status, may also be considered when evaluating breast cancer prognosis or diagnosis via the methods of the invention.

### Sample Source

In one embodiment of the present invention, breast cancer subtype is assessed through the evaluation of gene expression profiles of the intrinsic genes listed in Table 3 in one or more subject samples. The term subject, or subject sample, refers to an individual regardless of health and/or disease status. A subject can be a subject, a study participant, a control subject, a screening subject, or any other class of individual from whom sample is obtained and assessed in the context of the invention.

Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. Alternatively, a subject can be healthy with respect to any of the aforementioned factors or criteria. It will be appreciated that the term “healthy” as used herein, is relative to breast cancer status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer.

In particular embodiments, the methods for classifying breast cancer intrinsic subtypes include collecting a sample comprising a cancer cell or tissue, such as a breast tissue sample or a primary breast tumour tissue sample.

A “sample” or “biological sample” is intended to mean any sampling of cells, tissues, or bodily fluids in which expression of one or more intrinsic genes can be determined. Examples of such biological samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumour tissue sample. Biological samples may be obtained from a subject by a variety of techniques including, for example, by scraping or swabbing an area, by using a needle to aspirate cells or bodily fluids, or by removing a tissue sample (i.e., biopsy). Methods for collecting various biological samples are well known in the art. In some embodiments, a breast tissue sample is obtained by, for example, fine needle aspiration biopsy, core needle biopsy, or excisional biopsy. Fixative and staining solutions may be applied to the cells or tissues for preserving the specimen and for facilitating examination. Biological samples, particularly breast tissue samples, may be transferred to a glass slide for viewing under magnification. In one embodiment, the biological sample is a formalin-fixed, paraffin-embedded breast tissue sample, particularly a primary breast tumour sample.

### Gene Expression Profiling

In various embodiments, the present invention provides methods for classifying, treating, prognosing, diagnosing or monitoring breast cancer in subjects. In this embodiment, data obtained from analysis of intrinsic gene expression is evaluated using one or more pattern recognition algorithms. Such analysis methods may be used to form a predictive model, which can be used to classify test data. For example, one convenient and particularly effective method of classification employs multivariate statistical analysis modeling, first to form a model (a “predictive mathematical model”) using data (“modelling data”) from samples of known subtype to form a training set (e.g., from subjects known to have a particular breast cancer intrinsic subtype LumA, LumB, Basal-like, HER2-enriched, or normal-like), and second to classify an unknown sample (e.g., “testing set”) according to subtype.

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyze data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye.

The other approach is termed “supervised” whereby a training set of samples with known class or outcome is used to produce a computer-based or mathematical model which is then evaluated with independent validation data sets. Here, a “training set” of intrinsic gene expression data is used to construct a statistical model that predicts correctly the “subtype” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis.

The methods described herein are based on the gene expression profile for a plurality of subject samples using the intrinsic genes listed in Table 3. The plurality of samples includes a sufficient number of samples derived from subjects belonging to each subtype class. By “sufficient samples” or “representative number” in this context is intended a quantity of samples derived from each subtype that is sufficient for building a classification model that can reliably distinguish each subtype from all others in the group. A supervised prediction algorithm is developed based on the profiles of objectively-selected prototype samples for “training” the algorithm.

The generation of a gene expression score comprises calculating, for cells in the test data, the average (mean) read counts for each breast cancer intrinsic subtype Basal SC, HER2E SC, LumA SC and LumB SC. The cancer cells in the test sample are then assigned to the single-cell breast cancer intrinsic subtype with the highest signature score.

### Genes for Cell Determining Intrinsic Subtype

In some embodiments, at least about at least 20, 40, 60, 80, 100, 120, 140, 160, 180, 200, 220, 240, 260, 280, 300 or more of the genes listed in Table 3 are used to generate the gene expression profile. In other embodiments, at least 20, at least 30, at least 40, at least 50, at least 60, at least 70, or at least 80 of the intrinsic genes listed in Table 3 are used. In some embodiments, it is the combination of substantially all of the intrinsic genes that allows for the most accurate classification of intrinsic subtype and prognosis or determination of therapeutic response to treatment. Thus, in various embodiments, the methods disclosed herein encompass obtaining the genetic profile of substantially all the genes listed in Table 3. “Substantially all” may encompass at least 280, at least 290, at least 300, or all of the genes listed in Table 3.

It will also be understood by one of skill in the art that the subset of the genes listed in Table 3 can be used to predict breast cancer subtype or outcome. The same or another subset of the genes used to characterize an individual subject. In an embodiment, at least at least 20, 40, 60, 80, 100, 120, 140, 160, 180, 200, 220, 240, 260, 280, 300 or more of the genes listed in Table 3 are used to train the algorithm and at least at least 20, 40, 60, 80, 100, 120, 140, 160, 180, 200, 220, 240, 260, 280, 300 or more of the genes listed in Table 3 are used to characterize a subject.

“Gene expression” as used herein refers to the relative levels of expression and/or pattern of expression of a gene. The expression of a gene may be measured at the level of DNA, cDNA, RNA, mRNA, or combinations thereof “Gene expression profile” refers to the levels of expression of multiple different genes measured for the same sample. An expression profile can be derived from a biological sample collected from a subject at one or more time points prior to, during, or following diagnosis, treatment, or therapy for breast cancer (or any combination thereof), can be derived from a biological sample collected from a subject at one or more time points during which there is no treatment or therapy for breast cancer (e.g., to monitor progression of disease or to assess development of disease in a subject at risk for breast cancer), or can be collected from a healthy subject.

Gene expression profiles may be measured in a sample, such as samples comprising a variety of cell types, different tissues, different organs, or fluids (e.g., blood, urine, spinal fluid, sweat, saliva or serum) by various methods. Any methods available in the art for detecting expression of the intrinsic genes listed in Table 3 are encompassed herein. By “detecting expression” is intended determining the quantity or presence of an RNA transcript or its expression product of an intrinsic gene.

Methods for detecting expression of the intrinsic genes of the invention, that is, gene expression profiling, include methods based on hybridization analysis of polynucleotides, methods based on sequencing of polynucleotides, immunohistochemistry methods, and proteomics based methods. The methods generally detect expression products (e.g., mRNA) of the intrinsic genes listed in Table 3.

In embodiments, PCR-based methods, such as reverse transcription PCR (RT-PCR) (Weis et al., TIG 8:263-64, 1992), and array-based methods such as microarray (Schena et al., Science 270:467-70, 1995), preferably single-cell RNA sequencing, is used. By “microarray” is intended an ordered arrangement of hybridisable array elements, such as, for example, polynucleotide probes, on a substrate. The term “probe” refers to any molecule that is capable of selectively binding to a specifically intended target biomolecule, for example, a nucleotide transcript or a protein encoded by or corresponding to an intrinsic gene. Probes can be synthesized by one of skill in the art, or derived from appropriate biological preparations. Probes may be specifically designed to be labelled. Examples of molecules that can be utilized as probes include, but are not limited to, RNA, DNA, proteins, antibodies, and organic molecules.

Other methods for determining levels of cellular RNA may also be used in accordance with the invention including Nanostring GeoMX DSP platform that uses hybridisation of probes, followed by elution and sequencing of probes to estimate GE; Spatial transcriptomics (commercialised as visium by 10×genomics) which uses spotted arrays of barcoded capture probes to perform something similar to a microarray; and methods that use sequencing in situ to perform targeted RNA-Seq in situ.

Many expression detection methods use isolated RNA. The starting material is typically total RNA isolated from a biological sample, such as a tumour or tumour cell line, and corresponding normal tissue or cell line, respectively. If the source of RNA is a primary tumour, RNA (e.g., mRNA) can be extracted, for example, from frozen or archived paraffin embedded and fixed (e.g., formalin-fixed) tissue samples (e.g., pathologist-guided tissue core samples).

General methods for RNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RN easy mini-columns Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumour can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an intrinsic gene of the present invention, or any derivative DNA or RNA. Hybridization of an mRNA with the probe indicates that the intrinsic gene in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probes are immobilized on a solid surface and the mRNA is contacted with the probes, for example, in an Agilent gene chip array. A skilled person can readily adapt known mRNA detection methods for use in detecting the level of expression of the intrinsic genes of the present invention.

An alternative method for determining the level of intrinsic gene expression product in a sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87: 187 4-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers.

In particular aspects of the invention, intrinsic gene expression is assessed by quantitative RT-PCR. Numerous different PCR or QPCR protocols are known in the art and exemplified herein below and can be directly applied or adapted for use using the presently described compositions for the detection and/or quantification of the intrinsic genes listed in Table 3. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyders with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), !CYCLER® (Biorad Laboratories, Hercules, Calif.) and MX4000® (Stratagene, La Jolla, Calif.).

Quantitative PCR (QPCR) (also referred as realtime PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumour blocks, such as those used in archival tissue banks and routine surgical pathology specimens. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In another embodiment of the invention, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labelled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770, 358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts of cDNA clones are applied to a substrate in a dense array. The microarrayed genes, immobilized on the microchip, are suitable for hybridization under stringent conditions. Fluorescently labelled cDNA probes can be generated through incorporation of fluorescent nucleotides by reverse transcription of RNA extracted from tissues of interest. Labelled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual colour fluorescence, separately labelled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumour types.

### Data Processing

Illumina next-generation sequencing generates “raw” base-call bcl files. To computationally “demultiplex” the sequences to identify the source tumour and individual cells that each sequence read originates from, software methods, such as CellRanger from 10×Genomics, can be used. These sample demultiplexed sequence reads are also mapped to an appropriate reference genome.

To identify cells that reach certain quality control requirements, software methods, such as EmptyDrops from the DropletUtils package (doi: 10.1186/s13059-019-1662-y), and further features such as the percentage of mitochondrial reads in each cell, can be used.

It is often useful to pre-process single-cell gene expression data, for example, by addressing missing data, scaling, and normalization. Multivariate projection methods, such as principal component analysis (PCA), t-distributed stochastic neighbour embedding (tSNE), and uniform manifold approximation and projection (UMAP), are dimension reduction methods that are used to visualise and analyse gene expression profiles.

It is often useful to pre-process gene expression data, for example, by addressing missing data, translation, scaling, normalization, weighting, etc. Multivariate projection methods, such as principal component analysis (PCA), t-distributed stochastic neighbour embedding (tSNE), uniform manifold approximation and projection (UMAP), and partial least squares analysis (PLS), are so-called scaling sensitive methods. By using prior knowledge and experience about the type of data studied, the quality of the data prior to multivariate modelling can be enhanced by scaling and/or weighting. Adequate scaling and/or weighting can reveal important and interesting variation hidden within the data, and therefore make subsequent multivariate modelling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”).

“Translation” of the descriptor coordinate axes can be useful. Examples of such translation include normalization and mean centering. “Normalization” may be used to remove sample-to-sample variation. For microarray data, the process of normalization aims to remove systematic errors by balancing the fluorescence intensities of the two labelling dyes. The dye bias can come from various sources including differences in dye labelling efficiencies, heat and light sensitivities, as well as scanner settings for scanning two channels. Some commonly used methods or calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (Suppl.), 496-501). In one embodiment, the intrinsic genes disclosed herein can be normalized to control housekeeping genes. For example, the housekeeping genes described in U.S. Patent Publication 2008/0032293, which is herein incorporated by reference in its entirety, can be used for normalization. Exemplary housekeeping genes include MRPL19, PSMC4, SF3A1, PUM1, ACTB, GAPD, GUSB, RPLP0, and TFRC. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, microarray data is normalized using the LOWESS method, which is a global locally weighted scatterplot smoothing normalization function. In another embodiment, qPCR data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

In one embodiment, data is collected for one or more test samples and classified using the methods described herein. When comparing data from multiple analyses (e.g., comparing expression profiles for one or more test samples to the centroids constructed from samples collected and analyzed in an independent study), it will be necessary to normalize data across these data sets. In one embodiment, Distance Weighted Discrimination (DWD) is used to combine these data sets together (Benito et al. (2004) Bioinformatics 20(1):105-114, incorporated by reference herein in its entirety). DWD is a multivariate analysis tool that is able to identify systematic biases present in separate data sets and then make a global adjustment to compensate for these biases; in essence, each separate data set is a multidimensional cloud of data points, and DWD takes two points clouds and shifts one such that it more optimally overlaps the other.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

By way of example, the computer-based model that is produced by the training set of samples, as previously described, is stored in the computer readable medium. The computer-based model relates to gene expression signatures that define breast cancer intrinsic subtypes. A computer processor is configured to generate a test gene expression profile from cancer cells isolated from a test sample, wherein the test gene expression profile is based on expression of one or more of the genes, and to generate gene expression signature scores for the test gene expression profile, each gene expression signature score being a comparison between the test gene expression profile and the gene expression signature of a respective one of the breast cancer intrinsic subtype to which the computer-based model stored in the computer readable medium relates. The computer processor is further configured to classify the cancer cells from the test sample into one or more breast cancer intrinsic subtypes based on the gene expression signature score. The computer processor may also be configured to implement one or more of the other method steps described herein.

### Prognosis

Provided herein are methods for predicting breast cancer outcome within the context of the intrinsic subtype and optionally other clinical variables. Outcome or prognosis may refer to overall or disease-specific survival, event-free survival, or outcome in response to a particular treatment or therapy. In particular, the methods may be used to predict the likelihood of long-term, disease-free survival. Predicting the likelihood of survival of a breast cancer patient is intended to assess the risk that a patient will die as a result of the underlying breast cancer. Long-term, disease-free survival is intended to mean that the patient does not die from or suffer a recurrence of the underlying breast cancer within a period of at least five years, or at least ten or more years, following initial diagnosis or treatment.

In one embodiment, outcome is predicted based on classification of a subject according to subtype. This classification is based on expression profiling using one more of the intrinsic genes listed in Table 3. Generally, tumour subtype when classified according to the methods described herein is indicative of not only prognosis but also response to treatment.

In another embodiment, the methods described herein provide a measurement of the similarity of a test sample to all four subtypes which can be translated into a Risk Of Relapse (ROR) score that can be used in any patient population regardless of disease status and treatment options. The intrinsic subtypes and ROR also have value in the prediction of pathological complete response in women treated with, for example, neoadjuvant taxane and anthracycline chemotherapy. Thus, in various embodiments of the present invention, a ROR method model is used to predict outcome. Using these risk models, subjects can be stratified into low, medium, and high risk of relapse groups. Calculation of ROR can provide prognostic information to guide treatment decisions and/or monitor response to therapy.

In some embodiments described herein, the prognostic performance of the defined intrinsic subtypes and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., intrinsic gene expression profile with or without additional clinical factors, as described herein). The “hazard ratio” is the risk of death at any given time point for patients displaying particular prognostic variables. See generally Spruance et al., Antimicrob. Agents & Chemo. 48:2787-92, 2004.

The methods described herein can be trained for risk of relapse using subtype distances (or correlations) alone or using subtype distances with clinical variables as discussed supra. In one embodiment, the risk score for a test sample is calculated using intrinsic subtype distances alone using a suitable equation known in the art.

### Prediction of Response to Therapy

Breast cancer is managed by several alternative strategies that may include, for example, surgery, radiation therapy, hormone therapy, chemotherapy, or some combination thereof. As is known in the art, treatment decisions for individual breast cancer patients can be based on endocrine responsiveness of the tumour, menopausal status of the patient, the location and number of patient lymph nodes involved, estrogen and progesterone receptor status of the tumour, size of the primary tumour, patient age, and stage of the disease at diagnosis. Analysis of a variety of clinical factors and clinical trials has led to the development of recommendations and treatment guidelines for early-stage breast cancer by the International Consensus Panel of the St. Gallen Conference (2005). See, Goldhirsch et al., Annals Oneal. 16:1569-83, 2005. The guidelines recommend that patients be offered chemotherapy for endocrine non-responsive disease; endocrine therapy as the primary therapy for endocrine responsive disease, adding chemotherapy for some intermediate- and all high-risk groups in this category; and both chemotherapy and endocrine therapy for all patients in the uncertain endocrine response category except those in the low-risk group.

Stratification of patients according to risk of relapse and risk score disclosed herein provides an additional or alternative treatment decision-making factor. The methods comprise evaluating risk of relapse optionally in combination with one or more clinical variables, such as node status, tumour size, and ER status or any other clinical variables described herein or known in the art. The risk score can be used to guide treatment decisions. For example, a subject having a low risk score may not benefit from certain types of therapy, whereas a subject having a high risk score may be indicated for a more aggressive therapy.

The methods of the invention may find particular use in choosing appropriate treatment for early-stage breast cancer patients. The majority of breast cancer patients diagnosed at an early-stage of the disease enjoy long-term survival following surgery and/or radiation therapy without further adjuvant therapy. However, a significant percentage (approximately 20%) of these patients will suffer disease recurrence or death, leading to clinical recommendations that some or all early stage breast cancer patients should receive adjuvant therapy.

The methods of the present invention find use in identifying this high-risk, poor prognosis population of early-stage breast cancer patients and thereby determining which patients would benefit from continued and/or more aggressive therapy and close monitoring following treatment. For example, early-stage breast cancer patients assessed as having a high risk score by the methods disclosed herein may be selected for more aggressive adjuvant therapy, such as chemotherapy, following surgery and/or radiation treatment. In particular embodiments, the methods of the present invention may be used in conjunction with the treatment guidelines established by the St. Gallen Conference to permit practioners to make more informed breast cancer treatment decisions.

In various embodiments, the methods described herein provide information about breast cancer subtypes that cannot be obtained using standard clinical assays such as immunohistochemistry or other histological analyses. For example, subjects scored as estrogen receptor (ER)-positive and/or progesterone-receptor (PR)-positive would be indicated under conventional guidelines for endocrine therapy. For instance, the methods disclosed herein are capable of identifying a subset of these ER+/PgR+ cases that are classified as Basal-like, which may indicate the need for more aggressive therapy that would not have been indicated based on ER or PgR status alone.

Thus, the methods disclosed herein also find use in predicting the response of a breast cancer patient to a selected treatment. Predicting the response of a breast cancer patient to treatment is intended to mean assessing the likelihood that a patient will experience a positive or negative outcome with a particular treatment. As used herein, indicative of a positive treatment outcome refers to an increased likelihood that the patient will experience beneficial results from the selected treatment (e.g., complete or partial remission, reduced tumour size, etc.). Indicative of a negative treatment outcome is intended to mean an increased likelihood that the patient will not benefit from the selected treatment with respect to the progression of the underlying breast cancer.

In some embodiments, the relevant time for assessing prognosis or disease-free survival time begins with the surgical removal of the tumour or suppression, mitigation, or inhibition of tumour growth. In another embodiment, the risk score is calculated based on a sample obtained after initiation of neoadjuvant therapy such as endocrine therapy. The sample may be taken at any time following initiation of therapy, but is preferably obtained after about one month so that neoadjuvant therapy can be switched to chemotherapy in unresponsive patients. It has been shown that a subset of tumours indicated for endocrine treatment before surgery is non-responsive to this therapy. The model provided herein can be used to identify aggressive tumours that are likely to be refractory to endocrine therapy, even when tumours are positive for estrogen and/or progesterone receptors.

### Diagnosis and Treatment

In an aspect of the invention, there is provided methods for diagnosing and treating breast cancer in a subject.

The terms “patient” and “subject” to be treated herein are used interchangeably and refer to patients and subjects of human or other mammal and includes any individual being examined or treated using the methods of the invention. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., koalas, bears, wild cats, wild dogs, wolves, dingoes, foxes and the like).

The invention also provides a method of treating breast cancer. In some embodiments, the treatment may include any of those described herein or known in the art including surgery; chemotherapy; hormonal therapy; biological therapy such as immunotherapy, small molecule therapy or antibody therapy; and radiation therapy. In a further embodiment, the chemotherapy may include the administration of one or more of:
anthracyclines such as epirubicin (Pharmorubicin®), doxorubicin (Adriamycin®);
mitotic inhibitors such as taxanes, eg paclitaxel (Taxol®), docetaxel (Taxotere®);
antimetabolites such as 5-fluorouracil (5FU), capecitabine, 5-fluorouracil (5-FU), gemcitabine (Gemzar®);
alkylating agents such as cyclophosphamide;
taxanes such as paclitaxel (Taxol®), docetaxel (Taxotere®);
vinorelbine (Navelbine®); and
targeted therapies such as trastuzumab (Herceptin®), lapatinib (Tykerb®), bevacizumab (Avastin®).

In yet another embodiment, the radiotherapy may include the administration of one or more of:
3D conformal radiation therapy;
Intensity-modulated radiation therapy (IMRT);
Volumetric modulated radiation therapy (VMAT);
Image-guided radiation therapy (IGRT);
Stereotactic radiosurgery (SRS);
Brachytherapy;
Superficial x-ray radiation therapy (SXRT); and
Intraoperative radiation therapy (IORT).

In an embodiment, the subject to be treated exhibits one or more symptoms of a disease associated with breast cancer described herein or known in the art. Non-limiting examples may include one or more of:
presence of a lump in the breast or underarm;
thickening or swelling of part of the breast;
irritation or dimpling of breast skin;
redness or flaky skin in the nipple area or the breast;
pulling in of the nipple or pain in the nipple area;
nipple discharge including blood;
any change in the size or the shape of the breast; and
pain in an area of the breast.

Thus, a positive response to treatment with a therapeutically effective amount of any drug or compound identified herein may include amelioration of one of more of the above described symptoms or other symptoms known in the art. For instance, an individual having a positive response to treatment with any drug or compound administered as a result of the methods described herein may have a reduced presence of a lump in the breast or underarm or alternatively this may be surgically excised. An individual having a positive response to treatment with any drug or compound administered as a result of the methods described herein may also have reduced thickening or swelling, reduced irritation of breast skin, reduced redness or flaky skin in the nipple area or the breast, reduced nipple discharge or lessened pain or the symptoms may have disappeared altogether.

“Therapeutically effective amount” is used herein to denote any amount of a drug identified by the methods defined herein which is capable of reducing one or more of the symptoms associated with breast cancer. A single administration of the therapeutically effective amount of the drug may be sufficient, or they may be applied repeatedly over a period of time, such as several times a day for a period of days or weeks. The amount of the active ingredient will vary with the conditions being treated, the stage of advancement of the condition, the age and type of host, and the type and concentration of the formulation being applied. Appropriate amounts in any given instance will be readily apparent to those skilled in the art or capable of determination by routine experimentation.

The terms “treatment” or “treating” of a subject includes the application or administration of a drug or compound with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The invention also provides for methods for diagnosing a breast cancer clinical subtype in a test sample from a subject. Diagnosis as used herein refers to the determination that a subject or patient has a type of breast cancer, or intrinsic subtype of breast cancer as described herein or known in the art. The type of breast cancer diagnosed according to the methods described herein may be any type known in the art or described herein.

In an embodiment, one or more of the following additional diagnostic tests may be used in addition to the methods for diagnosis described herein. These include:
breast ultrasound: to create sonograms of areas inside the breast;
diagnostic mammogram or a screening mammogram or x-ray;
magnetic resonance imaging (MRI) to analyse areas inside the breast;
biopsy which may include removal of tissue or fluid from the breast to be looked at under a microscope and/or do more testing. The biopsy may be a fine-needle aspiration, core biopsy or open biopsy.

In an embodiment, the subject may exhibit one or more of the following risk factors: age, preferably over 50 years of age; genetic mutations to certain genes, such as BRCA1 and BRCA2; early menstrual periods before age 12 and starting menopause after age 55; having dense breasts; personal history of breast cancer or certain non-cancerous breast diseases; family history of breast or ovarian cancer; previous treatment using radiation therapy; or history of taking the drug diethylstilbestrol (DES).

In some embodiments, the subject diagnosed with breast cancer exhibits one or more of the symptoms of breast cancer described herein or known in the art.

### Pharmaceutical Compositions and Routes of Administration

The drugs or compounds that are provided herein that may be administered following the methods described herein may be provided in the form of a pharmaceutical composition comprising a therapeutically effective amount of any drug described herein or known in the art. In additional embodiments there is provided a pharmaceutical composition of any drug described herein or known in the art comprising a pharmaceutically acceptable salt.

The term “pharmaceutically acceptable salt” also refers to a salt of the compositions of the present invention having an acidic functional group, such as a carboxylic acid functional group, and a base. Pharmaceutically acceptable salts include, by way of non-limiting example, may include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycolate, heptanoate, hippurate, malate, hydroxymaleate, malonate, mandelate, mesylate, nicotinate, phthalate, teraphthalate, propiolate, propionate, phenylpropionate, sebacate, suberate, p-brornobenzenesulfonate, chlorobenzenesulfonate, ethylsulfonate, 2-hydroxyethylsulfonate, methylsulfonate, naphthiene-l-sulfonate, naphthalene-2-sulfonate, naphthiene-1,5 -sulfonate, xylenesulfonate, and tartarate salts.

Further, any drug described herein or known in the art can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxillary, stabilizing, thickening, lubricating, and colouring agents can be used.

In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In one embodiment, of any drug described herein or known in the art can take the form of solutions, suspensions, emulsion, drops, tablets, pills, pellets, capsules, capsules containing liquids, powders, sustained-release formulations, suppositories, emulsions, aerosols, sprays, suspensions, nanoparticles or microneedles or any other form suitable for use. In one embodiment, the composition is in the form of a capsule. Other examples of suitable pharmaceutical excipients are described in Remington's Pharmaceutical Sciences 1447-1676 (Alfonso R. Gennaro eds., 19th ed. 1995), incorporated herein by reference.

Where necessary, of any drug described herein or known in the art also includes a solubilizing agent. Also, the agents can be delivered with a suitable vehicle or delivery device as known in the art.

The of any drug described herein or known in the art can be co-delivered in a single delivery vehicle or delivery device. Compositions for administration can optionally include a local anaesthetic such as, for example, lignocaine to lessen pain at the site of the injection.

The of any drug described herein or known in the art may conveniently be presented in unit dosage forms and may be prepared by any of the methods well known in the art. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In one embodiment, of any drug described herein or known in the art is formulated in accordance with routine procedures as a composition adapted for a mode of administration described herein. In one aspect, the pharmaceutical composition is formulated for administration to the respiratory tract, the skin or the gastrointestinal tract. Accordingly, the pharmaceutical composition for administration to the respiratory tract may be formulated as an inhalable substance, such as common to the art and described herein. In another embodiment, the pharmaceutical composition for administration to the gastrointestinal tract may be formulated with an enteric coating, such as common to the art and described herein.

In an embodiment, the pharmaceutical composition may be administered in a single or as multiple doses. The pharmaceutical composition may be administered between one to three times in a 24 hour period, or daily over a 7 day period or longer. The frequency and timing of administration may be as known in the art.

Routes of administration include, for example: intradermal, intramuscular, intraperitoneal, intravenous, subcutaneous, intranasal, epidural, oral, sublingual, intracerebral, intra-lymph node, intratracheal, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and depends in-part upon the site of the medical condition. In most instances, administration results in the release of any agent described herein into the bloodstream.

In certain embodiments, the human suffering from or suspected of having breast cancer has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

### Kits

The present invention also provides kits useful for classifying breast cancer intrinsic subtypes and/or providing prognostic information. These kits comprise a set of capture probes and/or primers specific for the intrinsic genes listed in Table 3, as well as reagents sufficient to facilitate detection and/or quantitation of the intrinsic gene expression product. The kit may further comprise a computer readable medium.

In one embodiment of the present invention, the capture probes are immobilized on an array. By “array” is intended a solid support or a substrate with peptide or nucleic acid probes attached to the support or substrate. Arrays typically comprise a plurality of different capture probes that are coupled to a surface of a substrate in different, known locations.

The arrays of the invention comprise a substrate having a plurality of capture probes that can specifically bind an intrinsic gene expression product. The number of capture probes on the substrate varies with the purpose for which the array is intended. The arrays may be low-density arrays or high-density arrays and may contain 4 or more, 8 or more, 12 or more, 16 or more, 32 or more addresses, but will minimally comprise capture probes for the 50 intrinsic genes listed in Table 3.

Arrays may be packaged in such a manner as to allow for diagnostics or other manipulation on the device. See, for example, U.S. Pat. Nos. 5,856,174 and 5,922,591 herein incorporated by reference.

In another embodiment, the kit comprises a set of oligonucleotide primers sufficient for the detection and/or quantitation of each of the intrinsic genes listed in Table 3.

The oligonucleotide primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as discussed infra. The kit may further comprise reagents and instructions sufficient for the amplification of expression products from the genes listed in Table 3.

In order that the invention may be readily understood and put into practical effect, particular preferred embodiments will now be described by way of the following non-limiting examples.

### EXAMPLES

The present example illustrates an embodiment of the use of the methods described herein for subtyping tumour cells. In particular, this Example demonstrates a single cell method of intrinsic subtype classification (scSubtype) to identify recurrent neoplastic cell heterogeneity.

### Experimental Procedures

### Patient Material, Ethics Approval and Consent for Publication

Primary untreated breast cancers used in this study were collected under protocols x13-0133, x19-0496, x16-018 and x17-155. Human research ethics committee approval was obtained through the Sydney Local Health District Ethics Committee, Royal Prince Alfred Hospital zone, and the St Vincent's hospital Ethics Committee. Site-specific approvals were obtained for all additional sites. Written consent was obtained from all patients prior to collection of tissue and clinical data stored in a de-identified manner, following pre-approved protocols. Consent into the study included the agreement to the use of all patient tissue and data for publication. Two TNBC samples used for Visium analysis (1142243F and 1160920F) were sourced from BioIVT Asterand®.

### Tissue Dissociation

Samples collected in this study (Table 1) were analyzed from fresh surgical resections and cryopreserved tissue. Tumours were mechanically and enzymatically dissociated using Human Tumour Dissociation Kit (Miltenyi Biotec), following the manufacturer's protocol. For cryopreserved tissue, tumour tissues were thawed and washed twice with RPMI 1640 prior to dissociation, as previously described65. Following incubation at 37° C. for 30 to 60 min, the sample was resuspended in RPMI 1640 and filtered through MACS® SmartStrainers (70 μM; Miltenyi Biotec). The resulting single cell suspension was centrifuged at 300×g for 5 min. For fresh tissue processing, red blood cells were lysed with Lysing Buffer (Becton Dickinson) for 5 min and the resulting suspension was centrifuged at 300×g for 5 min. Where viability was <80%, viability enrichment was performed using the EasySep Dead Cell Removal (Annexin V) Kit (StemCell Technologies) as per manufacturer's protocol. Dissociated cells were resuspended in a final solution of PBS with 10% fetal calf serum (FCS) solution prior to loading on the 10×Chromium platform.

### Single-Cell RNA Sequencing Using 10×Chromium

Single-cell sequencing was performed using the Chromium Single-Cell v2 3′ and 5′ Chemistry Library, Gel Bead, Multiplex and Chip Kits (10×Genomics) according to the manufacturer's protocol. A total of 5,000 to 7,000 cells were targeted per well. Libraries were sequenced on the NextSeq 500 platform (Illumina) with pair-ended sequencing and dual indexing. A total of 26, 8 and 98 cycles were run for Read 1, i7 index and Read 2, respectively.

### Single-Cell RNA Sequencing Data Processing

Raw bcl files were demultiplexed and mapped to the reference genome GRCh38 using the Cell Ranger Single Cell v2.0 software (10×Genomics). The EmptyDrops method from the DropletUtils package (v1.2.2) (Tsai,et al., (2012) Cancer Cell 22, 725-36) was applied for cell filtering with additional cutoffs for cells with a gene and unique molecular identifier (UMIs) count greater than 200 and 250, respectively, and a mitochondrial percentage less than 20%. We used the Seurat v3.0.0 method (Stoeckius, M. et al., (2017) Nat Methods 14, 865-868) in R (v3.5.0) for data normalisation, dimensionality reduction and clustering using default parameters. Cell clusters were annotated using the Garnett method (Lim, E. et al., (2009) Nat Med 15, 907-13) (v0.1.4) with a classifier derived breast epithelial cell signatures (Aran et al., (2017) Genome Biol 18, 220), and immune and stromal cell types from XCell (Wagner, J. et al., (2019) Cell 177, 1330-1345 e18).

TABLE 1
Samples collected in this study.

| | | | | | | | | HER2 | |
|---|---|---|---|---|---|---|---|---|---|
| Case | | | | Cancer | | | HER2 | ISH | |
| ID | Gender | Age | Grade | Type | ER | PR | IHC | (ratio) | Ki67 |
| 3586 | Female | 43 | 3 | IDC | 100% 2-3+ | 100% 2-3+ | 3+ | Amplified | 30-50% |
| | | | | | | | | (6.8) | |
| 3838 | Female | 49 | 3 | IDC | 0 | 0 | 3+ | Amplified | 60% |
| | | | | | | | | (8.91) | |
| 3921 | Female | 60 | 3 | IDC | 0 | 0 | 3+ | Amplified | >50% |
| | | | | | | | | (10.46) | |
| 3941 | Female | 50 | 2 | IDC | 90% 3+ | 90% 3+ | 2+ | Non- | 10% |
| | | | | | | | | Amplified | |
| 3946 | Female | 52 | 3 | IDC | 0 | 0 | 0 | Non- | 60% |
| | | | | | | | | Amplified | |
| 3948 | Female | 82 | 3 | IDC | 90% 2-3+ | 80% 2+ | 0 | Non- | ~10% |
| | | | | | | | | Amplified | |
| 3963 | Female | 61 | 3 | IDC | 30% 1+ | 0 | 0 | Non- | 43% |
| | | | | | | | | Amplified | |
| 4040 | Female | 57 | 3 | IDC | 95% 3+ | 95% 2-3+ | 0 | Non- | >50% |
| | | | | | | | | Amplified | |
| 4066 | Female | 41 | 2 | IDC | 70% 3+ | 0 | 3+ | Amplified | 30% |
| | | | | | | | | (7.7) | |
| 4067 | Female | 85 | 2 | IDC | 100% 3+ | 95% 3+ | 1+ | Non- | 3-4% |
| | | | | | | | | Amplified | |
| 4290 | Female | 88 | 2 | IDC | 90% 3+ | 30% 2+ | 1+ | Non- | 10% |
| | | | | | | | | Amplified | |
| 4398 | Female | 52 | 3 | IDC | 95% 2+ | 80% 2+ | 2+ | Non- | 75% |
| | | | | | | | | Amplified | |
| 4404-1 | Female | 35 | 3 | IDC | 0 | 0 | 0 | Non- | 70% |
| | | | | | | | | Amplified | |
| 4461 | Female | 54 | 2 | IDC | 95% 3+ | ~5% 3+ | 2+ | Non- | 15% |
| | | | | | | | | Amplified | |
| 4463 | Female | 58 | 2 | IDC | 100% 2-3+ | 80% 2-3+ | 0 | Non- | 50% |
| | | | | | | | | Amplified | |
| 4465 | Female | 54 | 3 | IDC | 0 | 0 | 0 | Non- | 70% |
| | | | | | | | | Amplified | |
| 4471 | Female | 55 | 2 | ILC | 100% 3+ | 100% 3+ | 0 | Non- | 20% |
| | | | | | | | | Amplified | |
| 4495 | Female | 63 | 3 | IDC | 0 | 0 | 0 | Non- | 80% |
| | | | | | | | | Amplified | |
| 4497-1 | Female | 49 | 3 | IDC | 0 | 0 | 0 | Non- | 40% |
| | | | | | | | | Amplified | |
| 4499-1 | Female | 47 | 3 | IDC | 0 | 0 | 0 | Non- | 60-70% |
| | | | | | | | | Amplified | |
| 4513 | Female | 73 | 3 | MBC | 0 | 0 | 0 | Non- | 75% |
| | | | | | | | | Amplified | |
| 4515 | Female | 67 | 3 | IDC | 0 | 0 | 0 | Non- | 60% |
| | | | | | | | | Amplified | |
| 4517-1 | Female | 58 | 3 | IDC | 0 | 0 | 3+ | Amplified | 80% |
| 4523 | Female | 52 | 3 | MBC | 0 | 0 | 1+ | Non- | 90% |
| | | | | | | | | Amplified | |
| 4530 | Female | 42 | 2 | IDC | 95% 2+ | 95% 3+ | 1+ | Non- | 5% |
| | | | | | | | | Amplified | |
| 4535 | Female | 47 | 2 | ILC | 95% 3+ | 70% 2+ | 2+ | Non- | 10% |
| | | | | | | | | Amplified | |

| Case | Subtype | Treatment | | Notable Pathological | |
|---|---|---|---|---|---|
| ID | by IHC | status | Details of treatment | features | Stage |
| 3586 | HER2+/ | Naïve | — | Multifocal tumour with | pT(m)2, N2a |
| | ER+ | | | associatied high grade | |
| | | | | DCIS and extensive LVI | |
| 3838 | HER2+ | Naïve | — | Associated high grade | pT2, N1a |
| | | | | DCIS. | |
| 3921 | HER2+ | Naïve | — | Associated high grade | pT2, N2a |
| | | | | DCIS and focal LVI | (Stage IIIA) |
| 3941 | ER+ | Naïve | — | Multifocal tumour with | pT1c, N1a, |
| | | | | associated high grade | Mx |
| | | | | DCIS | |
| 3946 | TNBC | Naïve | — | Basal phenotype. | pT2, N0, |
| | | | | Reactive lymphoid | Mx |
| | | | | infiltrate with | |
| | | | | germinal centres. | |
| 3948 | ER+ | Naïve | — | Associated LCIS, with | pT2, N2a |
| | | | | LVI and perineural | |
| | | | | invasion | |
| 3963 | ER+ | Treated | AC, Paclitaxel, | Probable recurrence | pT2, pN0, |
| | | | Herceptin (administered | from 3 years prior | Mx, |
| | | | for Dx 3 years prior) | | Stage IIA |
| 4040 | ER+ | Naïve | — | Associated high grade | pT2, N0 |
| | | | | DCIS. | |
| 4066 | HER2+/ | Treated | Neoadjuvant AC | Associated high grade | pT2 N2a |
| | ER+ | | | DCIS and extensive LVI. | Mx |
| | | | | RCB-III, minimal or no- | |
| | | | | response to chemotherapy. | |
| 4067 | ER+ | Naïve | — | Associated low grade | pT2, N1(sn), |
| | | | | DCIS and focal | Mx |
| | | | | perineural invasion. | |
| 4290 | ER+ | Naïve | — | Locally advanced, skin | pT4b, Nx |
| | | | | and chest wall muscle | |
| | | | | involvement. | |
| 4398 | ER+ | Treated | Neoadjuvant FEC-D | Mixed morphology with | pT3, pN2a, |
| | | | | associated high grade | pMx, |
| | | | | DCIS, extensive LVI | Stage IIIA |
| | | | | and perineural invasion. | |
| | | | | RCB-III, minimal or no- | |
| | | | | response to chemotherapy. | |
| 4404-1 | TNBC | Naïve | — | Associated high grade | pT2, N1a, |
| | | | | DCIS and focal LVI. | Mx |
| 4461 | ER+ | Naïve | — | Associated intermediate | pT3, N1a, |
| | | | | to high grade DCIS, LVI | Mx |
| | | | | and perineural invasion. | |
| 4463 | ER+ | Naïve | — | IDC with areas of lobular- | pT3, N1, |
| | | | | like growth pattern, | Mx |
| | | | | but is E-cadherin positive. | |
| | | | | Associated low through | |
| | | | | high grade DCIS and LVI. | |
| 4465 | TNBC | Naïve | — | Basal phenotype - patchy | PT2, N0(sn) |
| | | | | CK5/6 and p63 positivity. | Mx |
| | | | | Associated high grade | |
| | | | | DCIS at periphery of | |
| | | | | tumour mass. | |
| 4471 | ER+ | Naïve | — | — | pT3, pN0 |
| | | | | | (i+) |
| 4495 | TNBC | Naïve | — | Medullary | pT1c, pN0 |
| | | | | features | |
| 4497-1 | TNBC | Naïve | — | Highly atypical cells | pT2, N1a, |
| | | | | with circumscribed | Mx |
| | | | | periphery, associated | |
| | | | | high grade DCIS and LVI. | |
| | | | | Accompanying lymphoid | |
| | | | | stroma. | |
| 4499-1 | TNBC | Naïve | — | BRCA2 mutation | |
| 4513 | TNBC | Treated | Neoadjuvant AC (4x), | Metaplastic, spindle cell | pT3, pN0, |
| | | | Paclitaxel (3x) | carcinoma with areas of | Mx, |
| | | | | sarcomatous appearance | Stage IIB |
| | | | | and inflammatory infiltrate. | |
| | | | | LVI present. RCB-II, | |
| | | | | partial pathological | |
| | | | | response to chemotherapy | |
| 4515 | TNBC | Naïve | — | Basal phenotype: | PpT1c, pN1, |
| | | | | CK5/6+ focal 40%, | Mi, |
| | | | | CK14+ focal 30%. | Stage IIA |
| | | | | Associated high grade | |
| | | | | DCIS and patchy | |
| | | | | lymphoid infiltrate. | |
| 4517-1 | HER2+ | Naïve | — | | |
| 4523 | TNBC | Treated | Neoadjuvant AC (4x), | Metaplastic carcinoma | pT2, pN0 |
| | | | Paclitaxel (1x) | with sebaceous | (i+), pM0, |
| | | | | differentiation. | Stage IIA |
| | | | | LVI present. RCB-II, | |
| | | | | partial pathological | |
| | | | | response to chemotherapy | |
| 4530 | ER+ | Naïve | — | Multifocal tumour with | pT3, pN3, |
| | | | | associated high grade | pMx, |
| | | | | DCIS and LVI. | Stage IIIA |
| 4535 | ER+ | Naïve | — | — | pT2, pN0 |
| | | | | | (i+), |
| | | | | | Stage IIB |

Identifying Neoplastic from Normal Breast Cancer Epithelial Cells

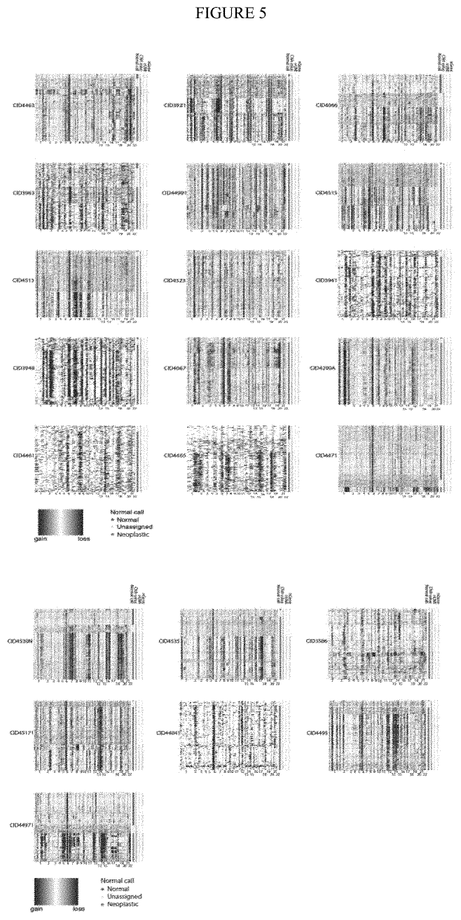

CNV signal for individual cells was estimated using the inferCNV method with a 100 gene sliding window. Genes with a mean count of less than 0.1 across all cells were filtered out prior to analysis, and signal was denoised using a dynamic threshold of 1.3 standard deviations from the mean Immune and endothelial cells were used to define the reference cell inferred copy-number profiles. Epithelial cells were used for the observations. Epithelial cells were classified into normal (non-neoplastic), neoplastic or unassigned using a similar method to that previously described by Neftel et al.31. Briefly, inferred changes at each genomic loci were scaled (between −1 and +1) and the mean of the squares of these values were used to define a genomic instability score for each cell. In each individual tumour, the top 5% of cells with the highest genomic instability scores were used to create an average CNV profile. Each cell was then correlated to this profile. Cells were plotted with respect to both their genomic instability and correlation scores. Partitioning around medoids (PAM) clustering was performed using the ‘pamk’ function in the R package ‘cluster’ to choose the optimum value for k (between 2-4) using silhouette scores, and the ‘pam’ function to apply the clustering. Thresholds defining normal and neoplastic cells were set at 2 cluster standard deviations to the left and 1.5 standard deviations below the first cancer cluster means. For tumours where PAM could not define more than 1 cluster, the thresholds were set at 1 standard deviation to the left and 1.25 standard deviations below the cluster means. This method was used to identify 27,506 neoplastic and 6084 normal cells in all tumours, the remaining 3208 cells were classed as unassigned (FIG. 5). Only tumours with at least 200 epithelial cells were used for this neoplastic cell classification step.

### Calling PAM50 on Pseudo-Bulks and Matching Bulk RNA-Seq

We constructed “pseudo-bulk” expression profiles for each tumour, where all the reads from all cells of a given tumour were added together, and then mapped as one sample. The resulting pseudo-bulk matrix thus constructed was named “Allcells-Pseudobulk” and was subsequently processed similarly to any bulk RNA-Seq sample (i.e. upper quartile normalized-log transformed) for calling molecular subtypes using the PAM50 method (Parker et al., (2009) J Clin Oncol 27, 1160-7). An important consideration made before PAM50 subtyping is to adjust a new sample set relative to the PAM50 training set according to their ER and HER2 status as detailed by Zhao et al (Zhao, et al (2015) Breast Cancer Res 17, 29). Thus, after ER/HER2 group-based adjustments, and then applying the PAM50 centroid predictor to the pseudo-bulk data, the methodology identified 7 of 20 Basal-like (CID3963, CID4465, CID4495, CID44971, CID4513, CID4515, CID4523), 4 of 20 HER2E (CID3921, CID4066, CID44991, CID45171), 5 of 20 LumA (CID3941, CID4067, CID4290A, CID4463, CID4530N), 3 of 20 LumB (CID3948, CID4461, CID4535) and 1 of 20 as Normal-like (CID4471).

We performed whole-transcriptome RNA-Seq using Ribosomal Depletion on 18 matching tumour samples from our single-cell dataset. RNA was extracted from diagnostic FFPE blocks using the High Pure RNA Paraffin Kit (Roche #03 270 289 001). The Sequence alignment was done using Salmon (Patro, et al., (2017) Nature Methods 14, 417-419 (2017). We then called PAM50 on each bulk tumour using Zhao et al (Zhao, et al (2015) Breast Cancer Res 17, 29) normalization and then the PAM50 centroid predictor (Table 2).

Intrinsic Subtype on scRNA-Seq Using scSubtype

To design and validate a new subtyping tool specific for scRNA-Seq data, we first divided our tumour samples into training and testing sets. The training dataset was defined by identifying tumours with unambiguous molecular subtypes. Here, we identified robust training set samples using two subtyping approaches: (i) PAM50 subtyping of the Allcells-Pseudobulk datasets (described above); and (ii) hierarchical clustering of the Allcells-Pseudobulk data with the 1,100 tumours in the TCGA BrCa RNA-Seq dataset using ˜2000 genes from an intrinsic breast cancer genelist. We first identified tumours that shared the same “concordant” subtype from both Allcells-Pseudobulk PAM50 calls and TCGA hierarchical clustering based subtype classifications (Table 2). Next, since our methodology aimed to subtype cancer cells, we removed any tumours with <150 cancer cells. Finally, we did not include cells from the two metaplastic samples (CID4513 and CID4523) in the training data because this is a histological subtype not used in the original PAM50 training set. Using this approach, we identified 10 tumour samples in the training dataset: HER2E (CID3921, CID44991, CID45171), Basal-like (CID4495, CID44971, CID4515), LumA (CID4290, CID4530) and LumB (CID3948, CID4535). Only tumour cells with greater than 500 UMIs were used for training and test datasets in scSubtype (total of 24,889 cells).

Within each training set subtype, we utilized the cancer cells from each tumour sample and performed pairwise single cell integrations and differential gene expression calculations. The integration was carried out in a “within group” pairwise fashion using the FindIntegrationAnchors and IntegrateData functions in the Seurat v3 package37. Briefly, the first step identifies anchors between pairs of cells from each dataset using mutual nearest neighbors. The second step integrates the datasets together based on a distance based weights matrix constructed from the anchor pairs. Differentially expressed genes were calculated between each pair using a Wilcoxon Rank Sum test by the FindAllMarkers function within Seurat v3. As the number of cancer cells per tumour sample were highly variable, this strategy prevented a bias of identifying genes for a training group from a sample with the highest number of cells. The following pairs were analyzed: HER2E (CID3921-CID44991, CID44991-CID45171, CID45171-CID3921), Basal-like (CID4495-CID44971, CID44971-CID4515, CID4515-CID4495), LumA (CID4290-CID4530) and LumB (CID3948-CID4535). In this way we identified unique upregulated genes per sample, but also genes broadly highlighting cells within each respective training group or subtype. We removed any duplicate genes occurring between the 4 training groups, which yielded 4 sets of genes composed of 89 genes defining Basal_SC, 102 genes defining HER2E_SC, 46 genes defining LumA_SC and 65 genes defining LumB_SC, which we define as “scSubtype” gene signatures (Table 3).

To assign a subtype call to a cell we calculated the average (i.e. mean) read counts for each of the 4 signatures for each cell. The SC subtype with the highest signature score was then assigned to each cell. We utilized this method to subtype all 24,489 neoplastic cells, from both our training samples (n=10) and the remaining test (n=10) set samples.

### Calculating Proliferation and Differentiation Scores

As previously described, we calculated the degree of epithelial cell differentiation status (DScore), and proliferation signature status, on each and every tumour cell in our scRNA-Seq cohort, as well as the 1,100 tumours in TCGA dataset. The 11 genes used to compute the proliferation signature status are independent of the scSubtype gene lists, while the Dscore is computed using a centroid based predictor with information from ˜20 thousand genes.

### Histology and Immunohistochemical Staining of CK5 and ER

Tumour tissue was fixed in 10% neutral buffered formalin for 24 hrs and then processed for paraffin embedding. Diagnostic tumour blocks were accessed for samples that did not have a research block available. Blocks were sectioned at 4 uM. Sections were stained with Haematoxylin and Eosin for standard histological analysis Immunohistochemistry (IHC) was performed on serial sections with pre-diluted primary antibodies against ER (clone 6F11; leica PA0151) or CK5 (clone XM26; leica PA0468) using suggested protocols on the BOND RX Autostainer (Leica, Germany). Antigen retrieval was performed for 20 min using BOND Epitope Retrieval solution 1 for ER or solution 2 for CK5, followed by primary antibody incubation for 60 min and secondary staining with the Bond Refine detection system (Leica). Slides were imaged using the Aperio CS2 Digital Pathology Slide Scanner.

### RESULTS

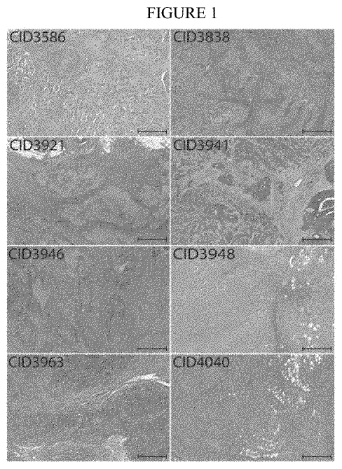

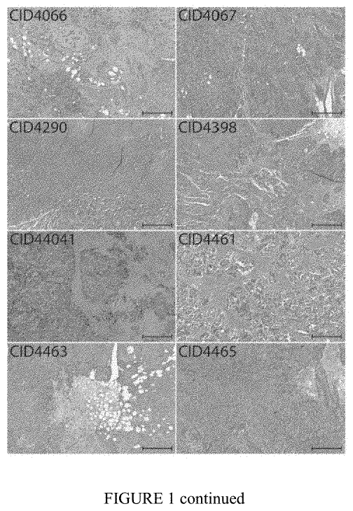

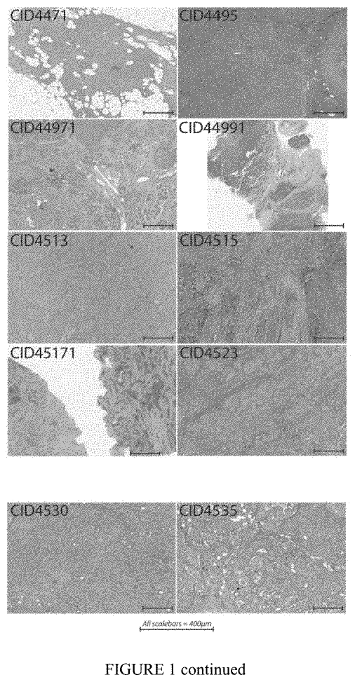

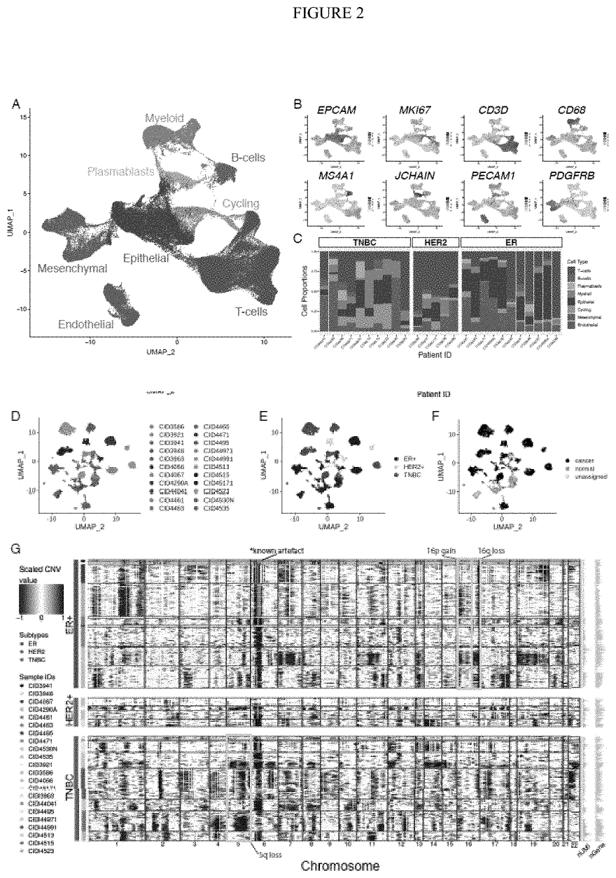

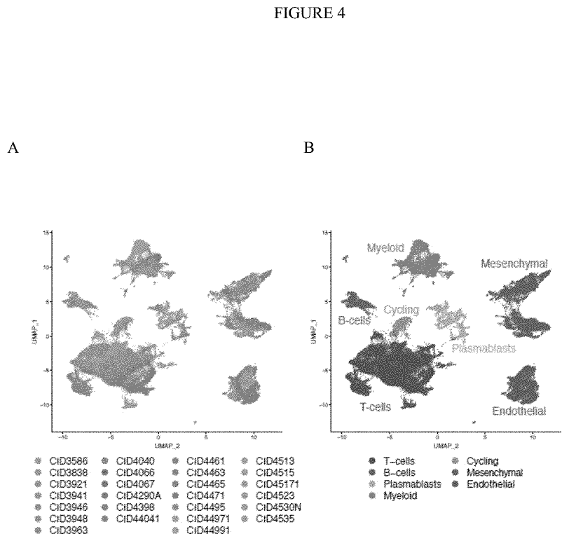

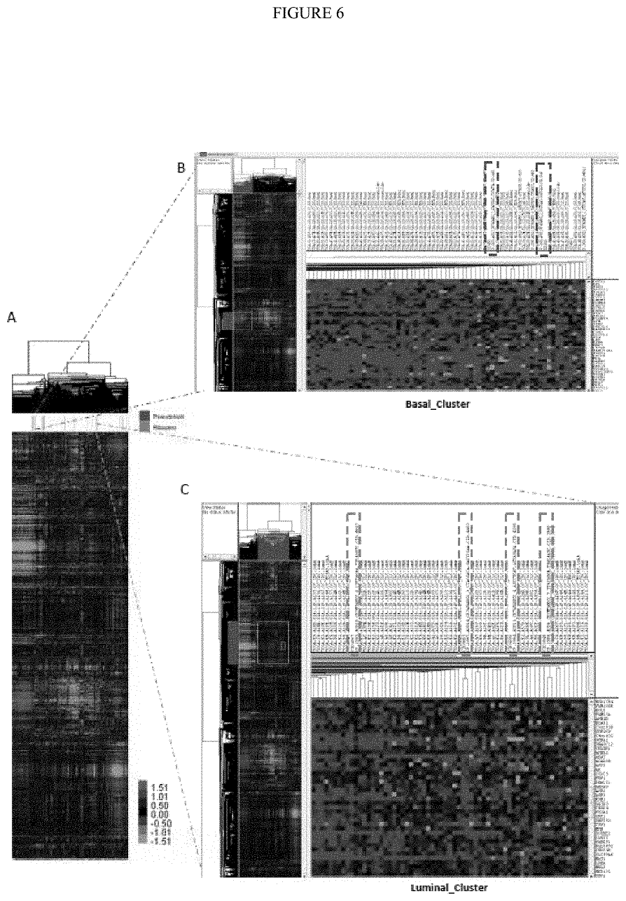

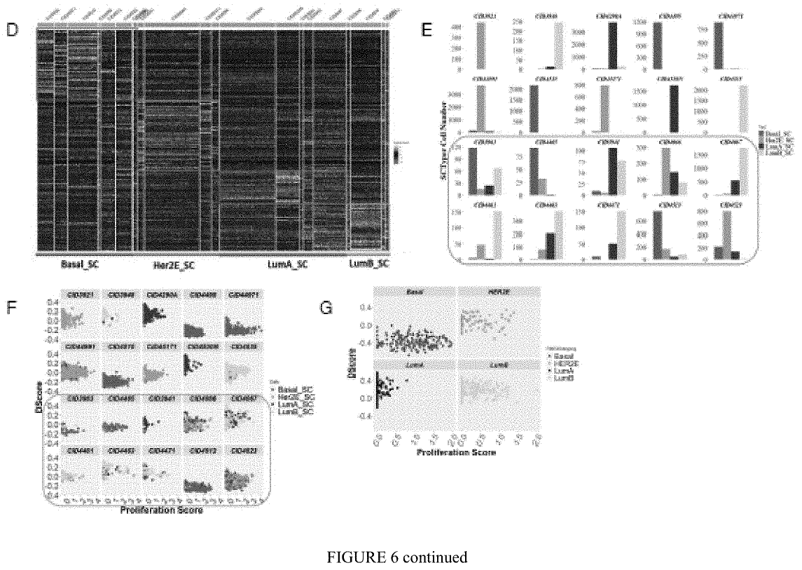

To elucidate the cellular architecture of BrCa, the inventors analysed 26 primary pre-treatment human BrCa, including 11 ER+, 5 HER2+ and 10 TNBCs, by scRNA-Seq (Table 1; FIG. 1). In total, 130,246 single-cells passed quality control (FIG. 4A-B) and were annotated using canonical lineage markers (FIG. 2A-B). These high-level annotations were further confirmed using published gene signatures. All major cell types were represented across all tumors and clinical subtypes of BrCa (FIG. 2C; FIG. 6E).

As previously reported in other cancer types, UMAP visualization showed a clear separation of epithelial cells by tumor, although three clusters contained cells from multiple patients and subtypes (FIG. 2D-E). We hypothesised that these were normal breast epithelial cells. In contrast, UMAP visualization of stromal and immune cells across tumors clustered together without batch correction (FIG. 6F). Since BrCa is largely driven by DNA copy number changes, we estimated single-cell copy number variant (CNV) profiles using InferCNV32 to distinguish neoplastic from normal epithelial cells (FIG. 2F-G). Cells confidently assigned as normal were re-clustered and annotated as one of the three main lineages of breast epithelia: myoepithelial, luminal progenitor and mature luminal. Within the neoplastic populations, we observed substantial levels of large-scale genomic rearrangement across a majority of cells (FIG. 2G; FIG. 5; Table 4). This revealed patient-unique copy number changes as well as those commonly seen in BrCa, such as chr1q and chr16p gain and chr16q loss in luminal cancers; and chr5q loss in ER− basal-like breast cancers.

As unsupervised clustering could not be used to find recurring neoplastic cell gene expression features between tumours, we asked whether we could classify cells using the established PAM50 method. Due to the inherent sparsity of single-cell data, we took the opportunity to develop a scRNA-Seq compatible method for intrinsic molecular subtyping. We constructed “pseudo-bulk” profiles from scRNA-Seq for each tumour, with at least 150 neoplastic cells, and applied the PAM50 centroid predictor. This identified 7 Basal-like, 4 HER2E, 5 LumA, 3 LumB and 1 Normal-like BrCa. To identify a robust training set, we used hierarchical clustering of the pseudo-bulk samples with the TCGA dataset of 1,100 BrCa using an ˜2,000 gene intrinsic BrCa genelist4 (FIG. 6A-C). Training samples were selected from those with concordance between pseudo-bulk PAM50 subtype calls and TCGA hierarchical clustering subtype classifications (Table 2).

With respect to Table 2, this Table shows a PAM50/scSubtype comparison of all patient samples included in the scSubtype analysis showing their clinical immunohistochemistry classification, PAM50 Subtype calls on pseudobulk RNA profiles from 10×scRNA-Seq and PAM50 Subtype calls on bulk RNA profiles using Ribozero mRNA-Seq data. Also, included are the number and percentage of individual neoplastic cells in each tumour assigned to each of the 4 scSubtype subtypes.

TABLE 2
PAM50/scSubtype comparison of patient samples.

| | | | | | | Concordance | Concordance | |
|---|---|---|---|---|---|---|---|---|
| | | | | | | between | between | |
| | | scRNA-Seq | Bulk | | | SCTyper and | SCTyper | |
| | | Allcells | RNA-Seq | | Majority | Allcells- | and Bulk | Basal_SC |
| Tumour | Clinical | Pseudobulk | (Ribozero) | SCTyper | SCTyper | Pseudobulk | RNA-Seq | cells |
| ID | IHC | PAM50 | PAM50 | dataset | Subtype | subtypes | subtypes | (freq) |
| CID3948 | ER | LumB | LumA | Training | LumB | Discordant | Discordant | 0 |
| CID4290A | ER | LumA | LumA | Training | LumA | Concordant | Concordant | 35 |
| CID4530N | ER | LumA | LumA | Training | LumA | Concordant | Concordant | 2 |
| CID4535 | ER | LumB | LumB | Training | LumB | Concordant | Concordant | 3 |
| CID3921 | HER2 | Her2 | Her2 | Training | Her2 | Concordant | Concordant | 0 |
| CID45171 | HER2 | Her2 | Not | Training | Her2 | Not | Not | 17 |
| | | | available | | | available | available | |
| CID4495 | TNBC | Basal | Basal | Training | Basal | Concordant | Concordant | 1183 |
| CID44971 | TNBC | Basal | Basal | Training | Basal | Concordant | Concordant | 882 |
| CID44991 | TNBC | Her2 | Not | Training | Her2 | Not | Not | 167 |
| | | | available | | | available | available | |
| CID4515 | TNBC | Basal | Basal | Training | Basal | Concordant | Concordant | 2167 |
| CID3941 | ER | LumA | LumA | Testing | LumA | Concordant | Concordant | 9 |
| CID4067 | ER | LumA | LumA | Testing | LumB | Concordant | Discordant | 15 |
| CID4461 | ER | LumB | LumB | Testing | LumB | Concordant | Concordant | 5 |
| CID4463 | ER | LumA | LumB | Testing | LumB | Discordant | Concordant | 2 |
| CID4471 | ER | Normal | Normal | Testing | Normal | Concordant | Concordant | 11 |
| CID3963 | HER2 | Basal | Basal | Testing | Basal | Concordant | Concordant | 116 |
| CID4066 | HER2_ER | Her2 | Normal | Testing | Her2 | Discordant | Discordant | 4 |
| CID4465 | TNBC | Basal | Basal | Testing | Basal | Concordant | Concordant | 91 |
| CID4513 | TNBC | Basal | LumB | Testing | Basal | Discordant | Discordant | 756 |
| CID4523 | TNBC | Basal | Basal | Testing | Her2 | Concordant | Discordant | 218 |

| | Her2e_SC | LumA_SC | LumB_SC | Basal_SC | Her2e_SC | LumA_SC | LumB_SC |
|---|---|---|---|---|---|---|---|
| Tumour | cells | cells | cells | cells | cells | cells | cells |
| ID | (freq) | (freq) | (freq) | (%) | (%) | (%) | (%) |
| CID3948 | 3 | 13 | 245 | 0 | 1.15 | 4.98 | 93.87 |
| CID4290A | 52 | 3748 | 218 | 0.86 | 1.28 | 92.47 | 5.38 |
| CID4530N | 1 | 1706 | 6 | 0.12 | 0.06 | 99.48 | 0.35 |
| CID4535 | 5 | 5 | 2210 | 0.13 | 0.22 | 0.22 | 99.42 |
| CID3921 | 441 | 0 | 0 | 0 | 100 | 0 | 0 |
| CID45171 | 792 | 1 | 3 | 2.09 | 97.42 | 0.12 | 0.37 |
| CID4495 | 0 | 1 | 0 | 99.92 | 0 | 0.08 | 0 |
| CID44971 | 6 | 4 | 2 | 98.66 | 0.67 | 0.45 | 0.22 |
| CID44991 | 3712 | 78 | 61 | 4.16 | 92.38 | 1.94 | 1.52 |
| CID4515 | 2 | 0 | 0 | 99.91 | 0.09 | 0 | 0 |
| CID3941 | 5 | 105 | 77 | 4.59 | 2.55 | 53.57 | 39.29 |
| CID4067 | 58 | 548 | 1731 | 0.64 | 2.47 | 23.3 | 73.6 |
| CID4461 | 47 | 3 | 152 | 2.42 | 22.71 | 1.45 | 73.43 |
| CID4463 | 81 | 198 | 378 | 0.3 | 12.29 | 30.05 | 57.36 |
| CID4471 | 0 | 50 | 151 | 5.19 | 0 | 23.58 | 71.23 |
| CID3963 | 15 | 24 | 67 | 52.25 | 6.76 | 10.81 | 30.18 |
| CID4066 | 294 | 144 | 79 | 0.77 | 56.43 | 27.64 | 15.16 |
| CID4465 | 32 | 1 | 0 | 73.39 | 25.81 | 0.81 | 0 |
| CID4513 | 167 | 49 | 86 | 71.46 | 15.78 | 4.63 | 8.13 |
| CID4523 | 795 | 134 | 20 | 18.68 | 68.12 | 11.48 | 1.71 |

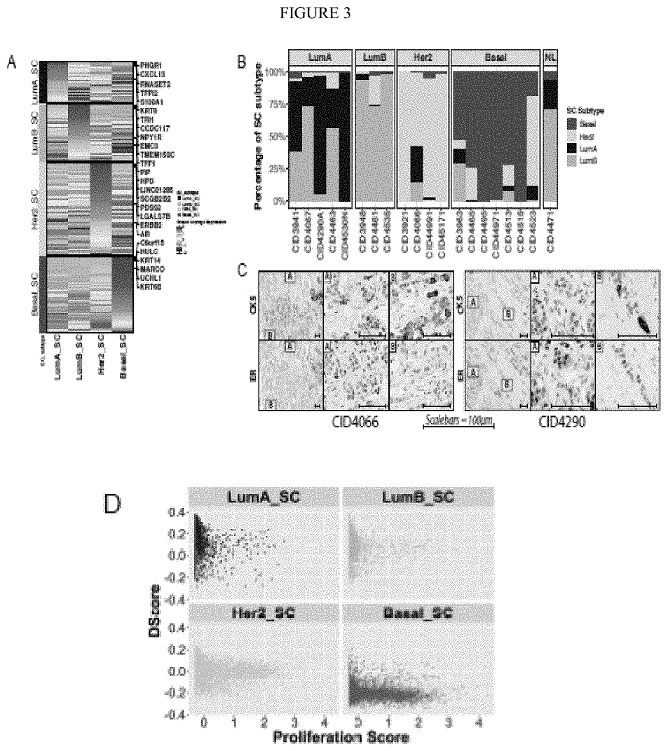

For each PAM50 subtype within the training dataset, we performed pairwise single cell integrations and differential gene expression to identify 4 sets of genes that would define our single-cell derived molecular subtypes (89 genes Basal_SC; 102 genes HER2E_SC; 46 genes LumA_SC; 65 genes LumB_SC; methods). We defined these genes as the “scSubtype” gene signatures (FIG. 3A; FIG. 6D; Table 3). Only four of these genes showed overlap with the original PAM50 gene list, including two from the Basal_SC set (ACTR3B and KRT14) and two from the Her2E_SC set (ERBB2 and GRB7). A subtype call for a given cell was based on the maximum scSubtype score. An overall tumour subtype was then assigned based on the largest population of cell subtypes (Table 2). This majority scSubtype approach showed 100% agreement with the PAM50 pseudo-bulk calls in the 10 training set samples and 66% agreement on the test set samples (FIG. 6E; Table 2). Of the 3 test set disagreements, two were LumA vs LumB, which are related profiles that may be hard to distinguish with a limited sample size, and the third was a metaplastic TNBC sample, which is a histological subtype not included in the original PAM50 training or testing datasets.

With respect to Table 3, this Table shows an scSubtype gene table where gene lists were used to define the single-cell scSubtype molecular subtype classifier, one for each scSubtype (Basal_SC, Her2E_SC, LumA_SC and LumB_SC).

TABLE 3
Genes used to define the single-cell scSubtype
molecular subtype classifier.

| Basal_SC | Her2E_SC | LumA_SC | LumB_SC |
|---|---|---|---|
| EMP1 | PSMA2 | SH3BGRL | UGCG |
| TAGLN | PPP1R1B | HSPB1 | ARMT1 |
| TTYH1 | SYNGR2 | PHGR1 | ISOC1 |
| RTN4 | CNPY2 | SOX9 | GDF15 |
| TK1 | LGALS7B | CEBPD | ZFP36 |
| BUB3 | CYBA | CITED2 | PSMC5 |
| IGLV3.25 | FTH1 | TM4SF1 | DDX5 |
| FAM3C | MSL1 | S100P | TMEM150C |
| TMEM123 | IGKV3.15 | KCNK6 | NBEAL1 |
| KDM5B | STARD3 | AGR3 | CLEC3A |
| KRT14 | HPD | MPC2 | GADD45G |
| ALG3 | HMGCS2 | CXCL13 | MARCKS |
| KLK6 | ID3 | RNASET2 | FHL2 |
| EEF2 | NDUFB8 | DDIT4 | CCDC117 |
| NSMCE4A | COTL1 | SCUBE2 | LY6E |
| LYST | AIM1 | KRT8 | GJA1 |
| DEDD | MED24 | MZT2B | PSAP |
| HLA.DRA | CEACAM6 | IFI6 | TAF7 |
| PAPOLA | FABP7 | RPS26 | PIP |
| SOX4 | CRABP2 | TAGLN2 | HSPA2 |
| ACTR3B | NR4A2 | SPTSSA | DSCAM.AS1 |
| EIF3D | COX14 | ZFP36L1 | PSMB7 |
| CACYBP | ACADM | MGP | STARD10 |
| RARRES1 | PKM | KDELR2 | ATF3 |
| STRA13 | ECH1 | PPDPF | WBP11 |
| MFGE8 | C17orf89 | AZGP1 | MALAT1 |
| FRZB | NGRN | AP000769.1 | C6orf48 |
| SDHD | ATG5 | MYBPC1 | HLA.DRB1 |
| UCHL1 | SNHG25 | S100A1 | HIST1H2BD |
| TMEM176A | ETFB | TFPI2 | CCND1 |
| CAV2 | EGLN3 | JUN | STC2 |
| MARCO | CSNK2B | SLC25A6 | NR4A1 |
| P4HB | RHOC | HSP90AB1 | NPY1R |
| CHI3L2 | PSENEN | ARF5 | FOS |
| APOE | CDK12 | PMAIP1 | ZFAND2A |
| ATP1B1 | ATP5I | TNFRSF12A | CFL1 |
| C6orf15 | ENTHD2 | FXYD3 | RHOB |
| KRT6B | QRSL1 | RASD1 | LMNA |
| TAF1D | S100A7 | PYCARD | SLC40A1 |
| ACTA2 | TPM1 | PYDC1 | CYB5A |
| LY6D | ATP5C1 | PHLDA2 | SRSF5 |
| SAA2 | HIST1H1E | BZW2 | SEC61G |
| CYP27A1 | LGALS1 | HOXA9 | CTSD |
| DLK1 | GRB7 | XBP1 | DNAJC12 |
| IGKV1.5 | AQP3 | AGR2 | IFITM1 |
| CENPW | ALDH2 | HSP90AA1 | MAGED2 |
| RAB18 | EIF3E | | RBP1 |
| TNFRSF11B | ERBB2 | | TFF1 |
| VPS28 | LCN2 | | APLP2 |
| HULC | SLC38A10 | | TFF3 |
| KRT16 | TXN | | TRH |
| CDKN2A | DBI | | NUPR1 |
| AHNAK2 | RP11.206M11.7 | | EMC3 |
| SEC22B | TUBB | | TXNIP |
| CDC42EP1 | CRYAB | | ARPC4 |
| HMGA1 | CD9 | | KCNE4 |
| CAV1 | PDSS2 | | ANPEP |
| BAMBI | XIST | | MGST1 |
| TOMM22 | MED1 | | TOB1 |
| ATP6V0E2 | C6orf203 | | ADIRF |
| MTCH2 | PSMD3 | | TUBA1B |
| PRSS21 | TMC5 | | MYEOV2 |
| HDAC2 | UQCRQ | | MLLT4 |
| ZG16B | EFHD1 | | DHRS2 |
| GAL | BCAM | | IFITM2 |
| SCGB1D2 | GPX1 | | |
| S100A2 | EPHX1 | | |
| GSPT1 | AREG | | |
| ARPC1B | CDK2AP2 | | |
| NIT1 | SPINK8 | | |
| NEAT1 | PGAP3 | | |
| DSC2 | NFIC | | |
| RP1.60O19.1 | THRSP | | |
| MAL2 | LDHB | | |
| TMEM176B | MT1X | | |
| CYP1B1 | HIST1H4C | | |
| EIF3L | LRRC26 | | |
| FKBP4 | SLC16A3 | | |
| WFDC2 | BACE2 | | |
| SAA1 | MIEN1 | | |
| CXCL17 | AR | | |
| PFDN2 | CRIP2 | | |
| UCP2 | NME1 | | |
| RAB11B | DEGS2 | | |
| FDCSP | CASC3 | | |
| HLA.DPB1 | FOLR1 | | |
| PCSK1N | SIVA1 | | |
| C4orf48 | SLC25A39 | | |
| CTSC | IGHG1 | | |
| | ORMDL3 | | |
| | KRT81 | | |
| | SCGB2B2 | | |
| | LINC01285 | | |
| | CXCL8 | | |
| | KRT15 | | |
| | RSU1 | | |
| | ZFP36L2 | | |
| | DKK1 | | |
| | TMED10 | | |
| | IRX3 | | |
| | S100A9 | | |
| | YWHAZ | | |

As another means of assessing the accuracy of scSubtype, we performed “true bulk” whole transcriptome RNA-Seq on 18 matching tumours in our scRNA-Seq cohort. As scSubtype does not include a Normal-like subtype, the two tumours called as Normal-like by RNA-Seq were not included in the comparison. We observed concordance between the majority scSubtype cell calls and the overall bulk tumour FFPE RNA-Seq profile in 12 of the remaining 16 BrCa, including 7 of the 8 matching training set tumours (Table 2). We also clustered the true bulk RNA-Seq data with TCGA and confirmed that the true bulk clustered with the pseudo-bulk profiles for 14 of 18 samples (FIG. 6A-C). These results highlight the strong concordance between our three methods of subtyping when applied across both bulk and scRNA-Seq datasets.

scSubtype revealed that 13 of 20 samples had less than 90% of neoplastic cells falling under one molecular subtype, while only one tumour (CID3921; HER2E) composed of neoplastic cells with a completely homogenous molecular subtype (FIG. 3B). For instance, in some luminal and HER2E tumours, scSubtype predicted small numbers of basal-like cells, which was validated by IHC in 2 cases. These two cases, which were clinically ER+, showed small pockets of morphologically malignant cells that were negative for ER and positive for cytokeratin-5 (CK5), a basal cell marker, among otherwise ER-positive tumour cells (FIG. 3C). The utility of scSubtype is further demonstrated by its ability to correctly assign a low cellularity lobular carcinoma (10% neoplastic cells; CID4471), evident both by histology (FIG. 1) and inferCNV (FIG. 5; Table 4), as a mixture of mostly LumB and LumA cells, which is consistent with the clinical IHC result. Bulk and pseudo-bulk RNA-Seq analyses incorrectly assigned CID4471 as a Normal-like tumour (Table 2), emphasizing the power of dissecting tumour biology at cellular resolution.

TABLE 4
Assignment of cells as neoplastic or non-neoplastic.

| sample_id | normal_cell_call | n |
|---|---|---|
| CID3586 | neoplastic | 50 |
| CID3586 | normal | 1017 |
| CID3586 | unassigned | 90 |
| CID3921 | neoplastic | 522 |
| CID3921 | normal | 16 |
| CID3921 | unassigned | 31 |
| CID3941 | neoplastic | 259 |
| CID3941 | normal | 2 |
| CID3941 | unassigned | 24 |
| CID3948 | neoplastic | 289 |
| CID3948 | normal | 7 |
| CID3948 | unassigned | 27 |
| CID3963 | neoplastic | 300 |
| CID3963 | normal | 36 |
| CID3963 | unassigned | 134 |
| CID4066 | neoplastic | 629 |
| CID4066 | normal | 343 |
| CID4066 | unassigned | 250 |
| CID4067 | neoplastic | 2476 |
| CID4067 | normal | 22 |
| CID4067 | unassigned | 179 |
| CID4290A | neoplastic | 4292 |
| CID4290A | normal | 72 |
| CID4290A | unassigned | 303 |
| CID44041 | neoplastic | 6 |
| CID44041 | normal | 211 |
| CID44041 | unassigned | 18 |
| CID4461 | neoplastic | 224 |
| CID4461 | normal | 0 |
| CID4461 | unassigned | 22 |
| CID4463 | neoplastic | 675 |
| CID4463 | normal | 56 |
| CID4463 | unassigned | 92 |
| CID4465 | neoplastic | 154 |
| CID4465 | normal | 54 |
| CID4465 | unassigned | 51 |
| CID4471 | neoplastic | 212 |
| CID4471 | normal | 2330 |
| CID4471 | unassigned | 318 |
| CID4495 | neoplastic | 1423 |
| CID4495 | normal | 15 |
| CID4495 | unassigned | 146 |
| CID44971 | neoplastic | 921 |
| CID44971 | normal | 1059 |
| CID44971 | unassigned | 259 |
| CID44991 | neoplastic | 4035 |
| CID44991 | normal | 137 |
| CID44991 | unassigned | 229 |
| CID4513 | neoplastic | 1519 |
| CID4513 | normal | 28 |
| CID4513 | unassigned | 115 |
| CID4515 | neoplastic | 2659 |
| CID4515 | normal | 50 |
| CID4515 | unassigned | 168 |
| CID45171 | neoplastic | 952 |
| CID45171 | normal | 8 |
| CID45171 | unassigned | 89 |
| CID4523 | neoplastic | 1241 |
| CID4523 | normal | 7 |
| CID4523 | unassigned | 103 |
| CID4530N | neoplastic | 1718 |
| CID4530N | normal | 565 |
| CID4530N | unassigned | 270 |
| CID4535 | neoplastic | 2950 |
| CID4535 | normal | 49 |
| CID4535 | unassigned | 290 |

To further support the validity of scSubtype, we calculated the degree of epithelial cell differentiation (DScore) and proliferation, both of which are independently associated with the molecular intrinsic subtype of each tumour cell (FIG. 3D; FIG. 6F). We also plotted the same for the 1,100 tumours of the TCGA dataset (FIG. 6G). Basal_SC cells tended to have low DScores and high proliferation scores whereas LumA_SC cells showed high DScores and low proliferation scores, as observed for whole tumours in TCGA.

To classify tumour cells in a manner consistent with the prior PAM50 bulk tumour classifier, we developed scSubtype, which was able to subtype tumours with low cellularity, for which bulk analysis had failed. Although heterogeneous expression of subtype markers (eg. cytokeratins, ER) has long been observed in BrCa, it was not known whether these were simply aberrations in marker expression or reflected functional diversity. scSubtype provides evidence for the latter, suggesting that intrinsic subtype heterogeneity exists within a majority of cancers. As for all classification methods, the performance of scSubtype will improve upon larger sample sizes applied to the training and test steps in future scRNA-Seq studies. Phenotypic diversity in cancer is generally associated with poorer outcomes. We hypothesize that intra-tumoural heterogeneity for intrinsic subtype may predict innate resistance to therapy and early relapse following therapy. For instance, the presence of basal-like or HER2-like cells in clinically luminal cancers (FIG. 3C) may cause early relapse following endocrine therapy.

1. A method for classifying cancer cells from a test sample into one or more breast cancer intrinsic subtypes, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3; and
d) generating gene expression signature scores for the test gene expression profile, each gene expression signature score being a comparison between the test gene expression profile and the gene expression signature of a respective breast cancer intrinsic subtype,
wherein the cancer cells from the test sample are classified into one or more breast cancer intrinsic subtypes based on the highest gene expression signature score, thereby classifying cancer cells from a test sample into one or more breast cancer intrinsic subtypes.

2. A method of generating gene expression signatures for classifying cancer cells into one or more breast cancer intrinsic subtypes, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal); and
b) generating, from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
wherein:
a test gene expression profile can be generated from cancer cells isolated from the test sample, wherein the test gene expression profile is based on expression of one or more of the genes listed in Table 3;
gene expression signature scores can be generated for the test gene expression profile, each gene expression signature score being a comparison between the test gene expression profile and the gene expression signature of a respective breast cancer intrinsic subtype; and
the cancer cells from a test sample can be classified into one or more breast cancer intrinsic subtypes based on the highest gene expression signature score.

3. A method for classifying cancer cells from a test sample into one or more breast cancer intrinsic subtypes, the method comprising:
a) generating a test gene expression profile from cancer cells isolated from the test sample, wherein the gene expression profile is based on expression of one or more of the genes listed in Table 3; and
b) generating gene expression signature scores for the test gene expression profile, each gene expression signature score being a comparison between the test gene expression profile and a gene expression signature of a respective breast cancer intrinsic subtype,
wherein:
a training gene expression profile can be generated from cancer cells that have been isolated from samples classified according to breast cancer intrinsic subtype Luminal A (LumA), Luminal B (LumB), Basal-like (Basal), HER2-enriched (HER2E), or Normal-like (Normal), and
the gene expression signatures can be generated from the training gene expression profile, the gene expression signatures defining breast cancer intrinsic subtypes Basal Single Cell (Basal SC), HER2-enriched Single Cell (HER2E SC), Luminal A Single Cell (LumA SC) and Luminal B Single Cell (LumB SC), each gene expression signature being based on expression of one or more of the genes listed in Table 3;
wherein the cancer cells from the test sample are classified into one or more breast cancer intrinsic subtypes based on the highest gene expression signature score.

4. The method according to any one of claims 1 to 3, wherein the generation of gene expression signatures from the training gene expression profile comprises using a machine learning algorithm, preferably a supervised algorithm.

5. The method according to any one of claims 1 or 3 to 4, wherein the method further comprises identifying a suitable treatment for the subject based on the classification of the cells in the test sample to the cancer intrinsic subtype.

6. The method according to claim 5, wherein the treatment comprises chemotherapy, hormonal therapy, radiation therapy, biological therapy such as immunotherapy, small molecule therapy or antibody therapy, or a combination thereof.

7. A method for diagnosing a breast cancer in a test sample from a subject, the method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample to form a testing set, wherein the test gene expression profile is based on expression of one or more of the genes listed in Table 3;
d) generating gene expression signature scores for the test gene expression profile, each gene expression signature score being a comparison between the test gene expression profile and the gene expression signature of a respective breast cancer intrinsic subtype,
wherein the cancer cells from the test sample are classified into one or more breast cancer intrinsic subtypes based on the highest gene expression signature score, and
wherein the proportions of cells isolated from the test sample and classified into the breast cancer intrinsic subtypes is determinative of the diagnosis of breast cancer in the subject,
thereby diagnosing a breast cancer in a subject.

8. The method according to claim 7, wherein the breast cancer is diagnosed as substantially HR+/HER2− (“Luminal A”); HR−/HER2− (“Triple Negative”); HR+/HER2+ (“Luminal B”) or HR−/HER2+ (“HER2-enriched”).

9. The method according to claim 7 or 8, wherein the subject has been diagnosed previously with a non-invasive or invasive carcinoma including ductal, lobular colloid (mucinous), medullary, micropapillary, papillary, and tubular invasive carcinoma.

10. The method according to any one of claims 1 to 9, wherein the sample was obtained from a subject exhibiting one or more of the following symptoms:
presence of a lump in the breast or underarm;
thickening or swelling of part of the breast;
irritation or dimpling of breast skin;
redness or flaky skin in the nipple area or the breast;
pulling in of the nipple or pain in the nipple area;
nipple discharge including blood;
any change in the size or the shape of the breast; and
pain in an area of the breast.

11. The method according to any one of claims 7 to 10, further comprising identifying a suitable treatment for the subject based on the diagnosis of the cancer intrinsic subtype.

12. The method according to claim 11, wherein the treatment comprises one or more treatments selected from the group consisting of surgery; chemotherapy; hormonal therapy; biological therapy such as immunotherapy, small molecule therapy or antibody therapy; and radiation therapy.

13. The method according to any one of claims 1 to 12, wherein the method further comprises one or more diagnostic tests selected from the list consisting of breast ultrasound, diagnostic mammogram, magnetic resonance imaging (MRI) or biopsy.

14. A method for prognosing breast cancer in a test sample from a subject, the method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) calculating a risk score for the cells of each of the samples and stratifying the risk scores into higher and lower risk groups;
d) generating a test gene expression profile from cancer cells isolated from the test sample, wherein the test gene expression profile is based on expression of one or more of the genes listed in Table 3;
e) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score;
f) generating a risk score for the cells isolated from the test sample based on the gene expression signature scores; and
g) determining whether the test sample falls within a higher or a lower risk group by comparing the risk score assigned in step (f) to the risk score assigned in (c), wherein assignment to a lower risk group indicates a more favourable outcome, and assignment to a higher risk group indicate a less favourable outcome,
thereby prognosing breast cancer in a test sample from a subject.

15. The method according to claim 14, wherein the prognosis is selected from the group comprising or consisting of breast cancer specific survival, event-free survival, or response to therapy.

16. A method for treating a breast cancer in a subject, the method comprising:
a) generating a training gene expression profile from cancer cells that have been isolated from samples classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3;
d) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score, and
e) administering a therapeutically effective amount of a treatment to the subject based on the breast cancer intrinsic subtype classification, thereby treating a breast cancer in the subject.

17. A method of predicting a response to a therapy in a test sample from a subject having breast cancer comprising classifying said subject according to a method comprising:
a) generating a training gene expression profile from cancer cells isolated from samples that have been classified according to breast cancer intrinsic subtype Luminal A (LumA), Luminal B (LumB), Basal-like (Basal), HER2-enriched (HER2E), or Normal-like (Normal);
b) generating from the training gene expression profile, gene expression signatures that define breast cancer intrinsic subtypes Basal Single Cell (Basal SC), HER2-enriched Single Cell (HER2E SC), Luminal A Single Cell (LumA SC) and Luminal B Single Cell (LumB SC), wherein each gene expression signature is based on expression of one or more of the genes listed in Table 3;
c) generating a test gene expression profile from cancer cells isolated from the test sample, wherein the test gene expression profile is based on expression of one or more of the genes listed in Table 3; and
d) generating gene expression signature scores for the test gene expression profile, each gene expression signature score being a comparison between the test gene expression profile and the gene expression signature of a respective breast cancer intrinsic subtype, wherein the cancer cells from the test sample are classified into one or more breast cancer intrinsic subtypes based on the highest gene expression signature score; and
wherein the intrinsic tumour subtype is indicative of response to the therapy,
thereby predicting a response to a therapy in a subject having breast cancer.

18. The method according to claim 17, wherein the therapy comprises an adjuvant or neoadjuvant therapy comprising radiotherapy, chemotherapy, immunotherapy, biological response modifiers or hormone therapy.

19. The method according to claim 17 or 18, further comprising a step of diagnosing the subject with breast cancer.

20. The method according to any one of claims 1 to 19, wherein the generation of a gene expression score comprises calculating the average read counts for each breast cancer intrinsic subtype Basal SC, HER2E SC, LumA SC and LumB SC, wherein the breast cancer intrinsic subtype with the highest signature score is assigned to each cell.

21. The method according to any one of claims 1 to 20, wherein the method further comprises providing or being provided with a test sample comprising cancer cells.

22. The method according to any one of claims 1 to 21, wherein cancer cells are isolated from the non-cancer cells, preferably by generating a CNV signal for individual cells.

23. The method according to any one of claims 1 to 22, wherein the test gene expression profile is generated from a sample comprising at least 200 cancer cells.

24. The method according to any one of claims 1 to 23, wherein the cancer cells are derived from a sample from a subject with an invasive carcinoma including ductal, lobular colloid (mucinous), medullary, micropapillary, papillary, tubular invasive carcinomas, preferably wherein the sample is derived from an untreated breast cancer.

25. The method according to any one of claims 1 to 24, wherein the method further comprises assessing one or more clinical variables including tumour size, node status, histologic grade, estrogen hormone receptor status, progesterone hormone receptor status, HER-2 levels, and tumour ploidy.

26. The method according to any one of claims 1 to 25, wherein the gene expression profile is generated using reverse transcription and real-time quantitative polymerase chain reaction (qPCR); microarray analysis, preferably single cell RNA-Seq.

27. The method according to any one of claims 1 to 26, wherein the gene expression profile is normalised to a control, preferably one or more housekeeping genes.

28. The method according to any one of claims 1 to 27, wherein the generation of the gene expression profile for the training set and testing set comprises determining expression of each of the genes listed in Table 3.

29. A kit for classifying a cancer intrinsic subtype in a test sample, the kit comprising reagents for the detection of the genes listed in Table 3.
